(19) United States
(12) Patent Application Publication
(10) Pub. No.: US 2018/0045715 A1
(43) Pub. Date: Feb. 15, 2018

(54) STRUCTURE AND FUNCTION OF THE SALICYCLIC ACID BINDING SITES ON HUMAN HMGB1 AND METHODS OF USE THEREOF FOR THE RATIONAL DESIGN OF BOTH SALICYCLIC ACID DERIVATIVES AND OTHER AGENTS THAT ALTER ANIMAL AND PLANT HMGBS ACTIVITIES

(71) Applicants: Boyce Thompson Institute for Plant Research, Inc., Ithaca, NY (US); RUTGERS, THE STATE UNIVERSITY OF NEW JERSEY, New Brunswick, NJ (US); Ospedale San Raffaele, Milano (IT)

(72) Inventors: Daniel F. Klessig, Dryden, NY (US); Sang-Wook Park, Auburn, AL (US); Hyong Woo Choi, Ithaca, NY (US); Frank C. Schroeder, Ithaca, NY (US); Gaetano T. Montelione, Highland Park, NJ (US); Keith Hamilton, Brick, NJ (US); Swapna Gurla, Skillman, NJ (US); Fei Song, Piscataway, NJ (US); Marco E. Bianchi, Milano (IT)

(21) Appl. No.: 15/551,448

(22) PCT Filed: Feb. 12, 2016
(86) PCT No.: PCT/US16/17651

§ 371 Date: Aug. 16, 2017

Related U.S. Application Data
(60) Provisional application No. 62/117,195, filed on Feb. 17, 2015.

(51) Int. Cl. G01N 33/50; G01N 24/08; G01R 33/46; C07K 14/52; A61K 36/31; A61K 31/19; A61K 49/14; G01N 33/566; A61K 38/17

(52) U.S. Cl. CPC G01N 33/5097; G01N 33/566; G01N 24/088; G01R 33/4633; A61K 38/177; A61K 36/31; A61K 31/19; A61K 49/14; C07K 14/52

(57) ABSTRACT

Compositions and methods for identifying agents which 1) mimic salicyclic acid binding to human, animal and plant high mobility group box proteins or 2) alter activities of these HMGBs by binding in or around their salicyclic acid-binding sites and agents so identified are disclosed.

### CROSS-REFERENCE TO RELATED APPLICATIONS

The present application claims the benefit of U.S. Provisional Patent Application No. 62/117,195, filed Feb. 17, 2015, filed May 30, 2008, the entire disclosure of which is incorporated by reference herein.

### STATEMENT REGARDING FEDERAL SPONSORED RESEARCH OR DEVELOPMENT

Pursuant to 35 U.S.C. § 202(c) it is acknowledged that the U.S. Government has rights in the invention described, which was made with funds from the National Institutes of Health, Grant Number, U54 GM094597 and the National Science Foundation, Grant Number IOS-0820405.

### FIELD OF THE INVENTION

This invention relates to the fields of medicine, medical science, agriculture, and the rational design of therapeutic agents. More specifically, the invention provides reagents useful for the design of salicylic derivatives analogs having therapeutic potential and design of compounds that alter the activities of High Mobility Group Box (HMGB) proteins. Such derivatives should have utility in humans, animals and different plant species.

### BACKGROUND OF THE INVENTION

Several publications and patent documents are cited throughout the specification in order to describe the state of the art to which this invention pertains. Each of these references are incorporated herein as though set forth in full. The plant-derived phenolic compound salicylic acid (SA) and its derivatives, known collectively as salicylates, have been widely used for thousands of years to reduce pain, fever, and inflammation (Vane, 1971; Vlot, Dempsey, & Klessig, 2009; Weissmann, 1991). Historical records from the third century B.C. indicate that Hippocrates prescribed the use of willow bark and leaves, which contains salicylates, to relieve pain and fever (Aboelsoud, 2010). The best-known salicylate is acetylsalicylic acid, commonly known as aspirin. In vivo aspirin rapidly undergoes hydrolysis to SA, which, like aspirin, has anti-inflammatory, antipyretic, and analgesic effects (Mitchell & Broadhead; 1967; Rowland et al.; 1967, Ekinci, 2011,). Additionally, aspirin's prophylactic use reduces the risk of heart attack, stroke, and certain cancers (Vlot et al., 2009; Rothwell et al., 2010; Armitage et al., 2012;). The primary mechanism of action of aspirin in mammals has been attributed to the disruption of eicosanoid biosynthesis through the irreversible inhibition via acetylation of cyclooxygenases (COX) 1 and 2, thereby altering the levels of prostaglandins (Ekinci, 2011). However, since SA has many of the same pharmacological effects as aspirin despite its poor ability to inhibit COX1/2 activity (Ekinci, 2011), aspirin/SA have additional molecular mechanisms of action that are only partially understood.

In plants, SA is involved in many physiological processes, including immunity, where it plays a central role (Vlot et al., 2009). To decipher SA's mechanisms of action, we have identified several plant SA-binding proteins (SABPs) (Vlot et al., 2009, Tian et al., 2012). By applying the approaches developed for identifying plant SABPs to mammalian cells, we have uncovered a new target of SA in humans, the high mobility group box 1 protein, HMGB1.

HMGB1 is an abundant, chromatin-associated protein that is present in all animal cells; fungi and plants have related proteins (Lotze & Tracey, 2005). Structurally, HMGB1 is composed of two basic DNA-binding domains, designated HMG boxes A and B, and a highly acidic C-terminal tail that participates in specific intramolecular interactions (Stottet al., 2010). In the nucleus, HMGB1 binds DNA to facilitate nucleosome formation and transcription factor binding (Celona et al., 2011). HMGB1 also acts as a damage-associated molecular pattern (DAMP) molecule with chemo-attractant and cytokine-inducing activities upon its release into the extracellular milieu from necrotic, damaged, or severely stressed cells (Andersson and Tracey, 2011).

Extracellular HMGB1 mediates a range of biological responses in association with multiple receptors, such as the Receptor for Advanced Glycation End products (RAGE), Toll-like receptor 2 (TLR2), TLR4, and C-X-C chemokine receptor type 4 (CXCR4) (Venereau et al., 2012). HMGB1 signaling through TLR2, TLR4 and RAGE leads to activation of NF-κB (Andersson and Tracey, 2011), whereas recognition of an HMGB1 complex with the chemokine CXCL12 by CXCR4 promotes the recruitment of inflammatory cells to damaged tissue (Schiraldi et al., 2012). HMGB1's diverse activities and receptors likely account for its multiple roles in human disease, including sepsis and arthritis (Yang et al., 2004; Schierbeck et al., 2011), atherosclerotic plaque formation (Porto et al., 2006), and cancer (Choi et al., 2003; Tang et al., 2010; Jube et al., 2012;). Consequently, HMGB1 has attracted considerable attention as an important drug target for various human diseases

The discovery that HMGB1binds SA, which inhibits its extracellular chemo-attractant activity provides new insights into the mechanisms through which salicylates manifest its anti-inflammatory and anti-cancer effects. Arabidopsis HMGB3 (AtHMGB1) also functions as a DAMP and binds SA. SA suppresses activation of MAPKs and deposition of callose induced by wild-type AtHMGB3, but not by an R50A/K54A mutant, suggesting HMGBs are conserved targets of SA.

### SUMMARY OF THE INVENTION

SA and several synthetic or natural derivatives bind human HMGB1 and inhibit its extracellular chemo-attractant and cytokine-inducing activities. The SA-binding sites in HMGB1 were identified by nuclear magnetic resonance (NMR) studies. Mutations in the SA-binding sites, which disrupt binding of SA and its derivatives, also suppress inhibition by SA and its derivatives of HMGB1's chemo-attractant activity. Both the synthetic and natural derivatives are more tightly bound by HMGB1 and as a result are more potent inhibitors of its pro-inflammatory activities, thus providing proof-of-concept that new molecules with enhanced efficacy against inflammation are attainable. Moreover, Arabidopsis HMGB3 bound SA, which suppressed its induction of plant immune responses. Mutation in the SA-binding site blocked SA binding and the resulting suppression of AtHMGB3's induction of immune responses.

Thus, the present invention provides a method for identifying agents which disrupt binding complexes formed between salicylic acid (SA) or derivatives thereof and high mobility group B1 (HMGB1) proteins. In one embodiment full length HMGB1 protein or protein fragments of HMGB1 having SA-binding sites in complex with SA are provided, wherein when said fragments are of human origin, they are selected from the group consisting of amino acids 1-165 (HMGB1ΔC), 8-78 (Box A), and 86-195 (Box B) (or construct variants with approximately these residue ranges) (e.g., + or − 5, 10, 15 or 20 amino acids). The complexes are contacted with a test agent, and the ability of the agent to displace SA or SA derivative from the HMGB1 binding complex is determined. In a preferred embodiment, the agent disrupts binding at at least one amino acid residue selected from the group consisting of Phe18, Thr22, Arg24, Glu25, His27, Lys28, Glu40, Cys45, Ser46, in the Box A domain of human HMGB1, or Phe103, Arg110, Lys114, Ser121, Gly123, Asp124 and Ala126 in the Box B domain of human HMGB1. The method can be used to screen for derivatives of SA which disrupt SA-HMGB1 binding complex formation, with the proviso that said agent is not glycyrrhizin. HMGB1 proteins can be tested from several species, including humans, animals and plants. In another embodiment of the invention, this method further comprises the step of measuring the effects of said agent on HMGB1 activity, said activity being selected from the group consisting of DNA binding, cytokine/chemokine-inducing activity, chemo-attractant activity, Cox-2-inducing activity, induction of autophagy, induction of angiogenesis, remodelling and repair of injured tissues. Agents using the method identified above are also within the scope of the invention.

In another aspect of the invention, a method for identifying human HMGB 1^{RE}-binding agents, which disrupt the formation of a binding complex between HMGB1-CXCL12 is provided. An exemplary method comprises incubating said complex in the presence and absence of said agent, agents which disrupt said complex relative to untreated controls having utility as HMGB1 modulating agents.

The invention also provides a method for identifying agents that slow the oxidation of the intramolecular disulfide bond formed between cysteine residues 23 and 45 of human HMGB1, said agents binding in or near the surface epitope of HMBG1 that includes at least one amino acid residue selected from the group consisting of residues Phe18, Thr22, Arg24, Glu25, His27, Lys28, Glu40, Cys45, and Ser46, Phe103, Arg110, Lys114, Ser121, Gly123, Asp124, and Ala126 in human HMGB1.

In yet another embodiment, a method for identifying agents that modulate the interaction between human HMGB1 and suppress the CXCL12/CXCR4 signaling pathway by binding in or near the surface epitope of HMBG1 at least one amino acid residue selected from the group consisting of residues Phe18, Thr22, Arg24, Glu25, His27, Lys28, Glu40, Cys45, and Ser46, Phe103, Arg110, Lys114, Ser121, Gly123, Asp124, and Ala126 is disclosed.

In another aspect, a method for identifying agents which disrupt binding complexes formed between salicylic acid (SA) or derivatives thereof and plant high mobility group proteins B1 and B3 (HMGB1 and HMGB3) is disclosed. An exemplary method entails providing a full length Arabidopsis thaliana HMGB1 or HMGB3 (AtHMGB1 or AtHMGB3) protein, or protein fragments of either protein, having SA-binding sites in complex with SA, contacting the complex with the agent, and determining whether the agent displaces said SA or said SA derivative from said binding complex. Agents which displace SA or said SA derivative being identified as analogs of SA which disrupt SA-AtHMGB1 and/or SA-AtHMGB3 binding complex formation, with the proviso that said agent is not glycyrrhizin.

Finally, the invention provides methods of use of agents which exhibit significantly enhanced potency when compared to SA. These include without limitation, acetyl-3-aminoethyl-salicyclic acid (ac-3AESA), amorfruitin A, amorfruitin 2, and amorfrutin B1. These agents can be used to advantage in methods which modulate HMGB1 activity, including, for example, DNA binding, cytokine/chemokine-inducing activity, chemo-attractant activity, Cox-2-inducing activity, induction of autophagy, induction of angiogenesis, and remodelling and repair of injured tissues.

The invention also provides a method for identifying agents that suppress AtHMGB1- or ATHMBG3-induced callose deposition in plant leaves.

### BRIEF DESCRIPTION OF THE DRAWINGS

FIG. 1A-FIG. 1G. HMGB1 binds SA. (FIG. 1A) Affinity purification of SABPs from human HeLa cells. Total protein extracts prepared from human HeLa cells were chromatographed on a SA-immobilized PharmaLink column. After washing with increasing concentrations of the biologically inactive SA analog, 4-hydroxy benzoic acid (4-HBA), the retained proteins were eluted with 5 mM SA, fractionated by SDS-PAGE and resolved by silver staining. Subsequent MS analyses identified band ‘a’ as HMGB1 (GI: 7669492). Note that the band denoted by ‘b’ was identified as soybean trypsin inhibitor, added by us during the process. MWM: molecular weight markers. (FIG. 1B) Photoaffinity labeling of HMGB1 using 4-azido-SA (4AzSA). HMGB1 was incubated with or without 0.1 mM 4AzSA in the absence or presence of the indicated concentrations of SA for 1 h, and then treated with UV light (50 mJ). Proteins labeled with 4AzSA were detected by immunoblot analysis with α-SA antibodies. Proteins stained with Ponceau S served as a loading control. (FIG. 1C) Evaluation of the SA-binding activity of various HMGB1 constructs by SPR. Sensorgrams of HMGB1^{RE} and HMGB1^{SS} (0.5 μM) interacting with 3AESA immobilized on the SPR sensor chip. (FIG. 1D) Schematic showing the residue boundaries of constructs of full-length (FL) HMGB1, HMGB1-ΔC, Box A and Box B used for SPR and NMR studies. (FIG. 1E) Sensorgrams of recombinant full-length (FL) HMGB1 (4 μM) and the HMGB1-ΔC construct (4 μM) interacting with 3AESA immobilized on the sensor chip. (FIG. 1F) Sensorgrams of recombinant Box A and Box B constructs of HMGB1 (4 μM) interacting with 3AESA immobilized on the sensor chip. Note the binding kinetics of Box A and Box B cannot be compared directly since the analysis of each was done on different sensor chips and on different days. (FIG. 1G) HMGB1 has strong affinity for3AESA with an apparent Kd of 1.48 nM. Sensorgrams of concentration-dependent HMGB1^{RE} interacting with 3AESA immobilized on the SPR sensor chip.

FIG. 2A-FIG. 2B. COX-2-independent SA inhibition of HMGB1^{RE}'s chemo-attractant activity. (FIG. 2A) Dose-dependent inhibition of HMGB1^{RE}'s chemo-attractant activity by SA. Migration of mouse NIH/3T3 fibroblast cells in the presence of 1 nM HMGB1^{RE}, but not of 0.1 nM fMLP, was blocked by SA. (FIG. 2B) SA suppressed HMGB1's chemo-attractant activity in mouse embryonic fibroblast cells knocked out for COX-2 (Ptgs2−/−). The data represent the mean±SD of three independent experiments. Different letters on top of the bars indicate significant difference analyzed by Tukey honest significant difference (HSD) test (P<0.05).

FIG. 3A-FIG. 3E. Identification of SA-binding sites on HMGB1. (FIG. 3A) ¹⁵N-¹H-HSQC spectra for HMGB1-ΔC was generated in the presence (blue) or absence (red) of 10 mM SA; expanded regions of the spectra were then superimposed. Residues that show significant chemical shift perturbations (CSPs) due to SA binding are labeled. (FIG. 3B) SA-induced CSPs mapped onto the 3D structure of human HMGB1 (2YRQ, residues 6-164). A space-filling model generated with the software PyMol (ref) is presented. The colors correspond to the amplitude of the observed CSPs (light blue: Δδ (N—H)<12 ppb; blue: 12<Δδ (N—H)<25 ppb; red: Δδ (N—H)>25 ppb, as indicated by the scale). Residues for which the backbone ¹⁵N-¹H resonance assignments are not available due to rapid exchange of surface amide protons and/or proline residues, are shown in white. (FIG. 3C) The ¹⁵N-¹H-HSQC spectra of HMBG1 and comparisons of chemical shift perturbations (CSPs) in full-length (FL) WT HMGB1, HMGB1-ΔC, Box A and Box B due to SA binding. ¹⁵N-¹H-HSQC spectra for FL WT HMBG1 was generated in the presence (blue) or absence (red) of 10 mM SA; expanded regions of the spectra were then superimposed. Residues that show significant CSPs due to SA binding are labeled. (FIG. 3D and FIG. 3E) CSPs (Δδ (N—H)) plotted as a function of residue number for three HMGB1 constructs. CSPs for the Box A construct are shown in green in (FIG. 3D), and those for the Box B construct are shown in brown in (FIG. 3E), while M (N—H) CSPs for the HMGB1-ΔC construct, shown in blue, are also plotted in both (FIG. 3D) and (FIG. 3E). Thresholds used for mapping CSPs onto the 3D structures are shown at 12 ppb (blue horizontal line) and 25 ppb (red horizontal line).

FIG. 4A-FIG. 4H. Ac3AESA and amorfrutin B1 are more potent inhibitors of HMGB1^{RE}'s chemo-attractant activity than SA. (FIG. 4A) Chemical structures of SA and its synthetic and natural derivatives. Conserved hydroxyl (—OH) and a carboxyl (—COOH) groups of salicylates are shown in blue color. (FIG. 4B) Dose-dependent inhibition of HMGB1^{RE}'s chemo-attractant activity by ac3AESA. Migration of NIH/3T3 fibroblast cells in the presence of 1 nM HMGB1^{RE}, but not of 0.1 nM fMLP, was blocked by ac3AESA. (FIG. 4C) Dose-dependent inhibition of HMGB1^{RE}'s chemo-attractant activity by amorfrutin B1 (amo B1). Migration of NIH/3T3 fibroblast cells in the presence of 1 nM HMGB1^{RE}, but not of 0.1 nM fMLP, was blocked by amo B 1. The data in (FIG. 4B) and (FIG. 4C) represent the mean±SD of three independent experiments. Different letters on top of the bars indicate significant difference analyzed by Tukey HSD test (P<0.05). (FIG. 4D-FIG. 4F) ¹⁵N-¹H Δδ(N—H) CSPs due to SA (FIG. 4D), ac3AESA (FIG. 4E), and amorfrutin B1 (FIG. 4F) binding are mapped onto the 3D structure of human HMGB1 (2YRQ, residues 6-164), which is oriented to highlight the similarities and differences in the SA-binding sites of Box A and Box B. Residues with significant Δδ(N—H) CSPs (>25 ppb) are shown in red and labelled, and the residues that both exhibit significant Δδ(N—H) CSPs and were mutated to Ala are shown green. 2D ¹⁵N-¹H HSQC spectra for full-length (FL) HMGB1. Spectra were recorded in the absence (black) or presence of 3 mM ac3AESA (blue) (FIG. 4G), or 3 mM amorfrutin B1 (green) (FIG. 4H). Expanded regions of the spectra were then superimposed. Residues that show significant CSPs due to ligand binding are labeled.

FIG. 5A-FIG. 5B. HSQC Titration experiment for K_{d} measurement of salicylic acid (SA) binding to HMGB1 Box A domain. (FIG. 5A) 2D ¹⁵N-¹H HSQC spectra at 600 MHz for 0.2 mM HMGB1 Box A. Spectra were recorded in the absence (blue) or presence of of 0.5 mM (red), 1 mM (cyan), 3 mM (red), 9 mM (green) and 12 mM (purple) SA. Expanded regions of the spectra were then superimposed. Residues with significant CSPs that are used for K_{d} fitting are labeled. (FIG. 5B) The solid curve represents the best fit solution of the equation that describes 1:1 complex formation. The mean K_{d}=90 mM and mean CSPₘₐₓ=0.5 ppm

FIG. 6A-FIG. 6B HSQC Titration experiment for K_{d} measurement of acetyl-3-aminoethyl-salicyclic acid (ac3AESA) binding to HMGB1 Box A domain. (FIG. 6A) 2D ¹⁵N-¹H HSQC spectra for 0.2 mM HMGB1 Box A. Spectra were recorded in the absence (light blue) or presence of of 0.5 mM (red), 0.8 mM (green), 1 mM (Magenta), 2 mM (yellow), 4 mM (blue) and 8 mM (purple) ac3AESA. Expanded regions of the spectra were then superimposed. Residues with significant CSPs that are used for K_{d} fitting are labeled. (FIG. 6B) The solid curve represents the best fit solution of the equation that describes 1:1 complex formation. The mean K_{d}=43.5 mM and mean CSPₘₐₓ=0.57 ppm

FIG. 7. Waterlogsy experiments demonstrating SA binding to full-length HMGB1, CXCL12, and the HMGB1-CXCL12 Complex. Ligand-detected WaterLogsy experiments using 6 mM SA and 20 μM HMGB1 (black), CXCL12 (orange) or HMGB1/CXCL12 complex (purple). The top green trace shows the waterLOGSY spectrum of free SA, in which the labeled numbers indicate the resonances assignments to protons in aromatic ring (positions 3, 4, 5, and 6) of SA (shown in insert). These results demonstrate that SA binds CXCL2, HMGB1, and the complex formed between CXCL12 and HMGB1.

FIG. 8. Waterlogsy experiments demonstrating ac-3AE-SA binding to full-length HMGB1, CXCL12, and the HMGB1-CXCL12 Complex. Ligand-detected WaterLogsy experiments using 5 mM ac3AESA and 20 μM HMGB1 (black), CXCL12 (orange) or HMGB1/CXCL12 complex (purple). The top green trace shows the waterLOGSY spectrum of free ac3AESA, in which the labeled numbers indicate the resonances assignments to protons in aromatic ring (positions 4, 5, and 6) and acetyl-amide proton (7) of ac3AESA (shown in insert). These results demonstrate that ac3AESA binds CXCL2, HMGB1, and the complex formed between CXCL12 and HMGB1.

FIG. 9A-FIG. 9B. Saturation transfer difference (STD) experiments demonstrating that ac3AESA has stronger binding affinity to full-length HMGB1 than SA. Ligand-detected STD experiments using 6 mM SA (FIG. 9A) or a titration using 0.6 to 5.8 mM ac3AESA (FIG. 9B), with 20 μM HMGB1 (black). In each panel, the top trace is the spectrum of the ligand itself (SA in the top panel, and ac3AESA in FIG. 9B), and the traces below these are the STD spectra with various amounts of ligand added. These results demonstrate a STD effect for HMGB1 binding by ac-3AESA binding HMGB1 at concentrations greater than about 1 mM, but no STD effect for HMGB1 binding by SA at concentrations as high as 6 mM, indicating that HMGB1 is binding ac3AESA more tightly than to SA.

FIG. 10A-FIG. 10G. Arg-24 and Lys-28 are required for binding SA and its derivatives and for their inhibition of HMGB 1^{RE}'s chemo-attractant activity. (FIG. 10A) Inhibition of wild-type (WT) and mutant (R24A/K28A) HMGB1^{RE}'s chemo-attractant activity by SA, ac3AESA, and amorfrutin B1. R24A/K28A's chemo-attractant activity was not inhibited by SA or ac3AESA, and only partially by amorfrutin B1. The data represent the mean±SD of three independent experiments. Different letters on top of the bars indicate significant difference analyzed by Tukey HSD test (P<0.05). (FIG. 10B-FIG. 10E) Mutant HMGB1 does not bind mutant SA. (FIG. 10B) ¹⁵N-¹H HSQC spectra for WT HMGB1 (˜0.1 mM) were generated in the presence (blue) or absence (red) of 15 mM SA. (FIG. 10C) Residues that show significant CSPs due to SA binding are labeled in expanded regions of the superimposed spectra. (FIG. 10D) ¹⁵N-¹H HSQC spectra for R24A/K28A (˜0.1 mM) were generated in the presence (green) or absence (red) of 15 mM SA. (FIG. 10E) Residues that show significant CSPs due to SA binding of WT, but not of R24A/K28A, are labeled in the expanded regions of the superimposed spectra. (FIG. 10F) Comparison of 3D structure of WT vs R24A/K28A mutant HMGB1 by ¹⁵N-¹H HSQC 2D NMR spectroscopy. R24A/K28A has a 3D structure similar to the WT HMGB1. This is demonstrated by superimposed ¹⁵N-¹H HSQC spectra of WT HMGB1 (blue) and of R24A/K28A (red). Most NH sites exhibit little or no CSP due to this double mutation. (FIG. 10G) Amide NH's that show significant CSPs in R24A/K28A (red) compared to the WT (blue) are all localized in or near the SA-binding site in Box A, and include the NH sites that exhibit CSP due to SA binding, which are labeled in this expanded region of the superimposed spectra.

FIG. 11A-FIG. 11H. Ac3AESA and amorfrutin B1 do not bind full-length (FL) R24A/K28A HMGB1. (FIG. 11A) ¹⁵N-¹H HSQC spectra for FL WT HMGB1 (˜0.1 mM) were generated in the presence (blue) or absence (red) of 3 mM ac3AESA. (FIG. 11B) Residues that show significant CSPs due to ac3AESA binding are labeled in expanded regions of the superimposed spectra. (FIG. 11C) ¹⁵N-¹H HSQC spectra for R24A/K28A (˜0.1 mM) were generated in the presence (green) or absence (red) of 3 mM ac3AESA. (FIG. 11D) Residues that show significant CSPs due to ac3AESA binding of WT, but not of R24A/K28A, are labeled in the expanded regions of the superimposed spectra. (FIG. 11E) Amorfrutin B1 does not bind full-length (FL) R24A/K28A HMGB1. ¹⁵N-¹H HSQC spectra for FL WT HMGB1 (˜0.1 mM) were generated in the presence (blue) or absence (red) of 3 mM amorfrutin B 1. (FIG. 11F) Residues that show significant CSPs due to amorfrutin B1 binding are labeled in expanded regions of the superimposed spectra. (FIG. 11G) ¹⁵N-¹H HSQC spectra for R24A/K28A (˜0.1 mM) were generated in the presence (green) or absence (red) of 3 mM amorfrutin B 1. (FIG. 11H) Residues that show significant CSPs due to amorfrutin B1 binding of WT, but not of R24A/K28A, are labeled in the expanded regions of the superimposed spectra.

FIG. 12. (I) SA inhibition of chemotaxis of putative SA-binding mutants of HMGB1^{RE}: R24A/K28A, H27A/R48A, and K12A/K68A. Putative SA-binding sites were predicted based on the ¹⁵N-¹H HSQC 2D NMR spectroscopy and SwissDock, a protein-small molecule docking web service (http://www.swissdock.ch). Migration of NIH/3T3 fibroblast cells induced by 1 nM wild-type (WT) and mutant HMGB1^{RE} in the absence or presence of 30 μM SA. The data represent the mean±SD of three independent experiments.

FIG. 13A-FIG. 13B. Effects of salicylates on HMGB1^{SS}'s cytokine-inducing activities in human macrophages. (FIG. 13A) Dose-dependent (left panels) and time-course (right panels) expression of COX-2 and inflammatory cytokine genes (IL-6 and TNFα) by HMGB1^{SS} in human macrophages. For dose-dependent experiment, macrophages were activated for 3 h with the indicated concentrations of HMGB1^{SS}. For time-course experiments, 10 μg/ml HMGB1^{SS} (˜300 nM) or 10 ng/ml LPS were used. (FIG. 13B) Inhibition of HMGB 1^{SS}'s, but not of LPS's, cytokine-inducing activity by SA and ac3AESA in human macrophages. Human macrophages were activated for 3 h with 1 μg/ml HMGB1^{SS} (˜30 nM) (left panels) or 10 ng/ml LPS (right panels) in the absence or presence of 100 μM SA or 1 μM ac3AESA. The data are the mean±SD of three independent experiments. Different letters on top of the bars indicate significant difference analyzed by Tukey HSD test (P<0.05).

FIG. 14. Schematic of SA's mechanisms of suppression of DAMP activity of HMGB proteins In animal cells. Extracellular HMGB 1 is recognized by multiple receptors, including CXCR4 and TLR4 (Schiraldi et al., 2012; Venereau et al., 2012). The reduced form of HMGB1 (HMGB1^{RE}) interacts with CXCL12 to induce CXCR4-mediated cell migration. SA inhibits heterocomplex formation between HMGB1^{RE} and CXCL12, thereby inhibiting cell migration induced by HMGB1^{RE}. Recognition of disulfide-bonded forms of HMGB1 (HMGB1^{SS}) by TLR4 results in translocation of NF-κB into the nucleus (Venereau et al., 2012), which leads to transcriptional activation of Cox-2 and the pro-inflammatory cytokine genes, IL-6 and TNF-α. SA suppresses HMGB1^{SS}-induced Cox-2, IL-6, and TNF-α expression. In plant, the receptor for AtHMGB3 has not yet been identified. Extracellular AtHMGB3 activates MAPKs MPK3 and MPK6 and induces callose deposition, which depends on the kinases BAK1 and BKK1, both of which are essential for DAMP-mediated plant immunity (Schwessinger et al., 2011). SA specifically inhibits induction by AtHMGB3 of these plant immune responses.

FIG. 15A-FIG. 15I. SA binding by AtHMGB3 and inhibition of its DAMP activity. (FIG. 15A) Photoaffinity labeling of AtHMGB3 using 4AzSA. AtHMGB3 was incubated with or without 50 μM 4 AzSA in the absence or presence of the indicated concentrations of SA for 1 h, and then irradiated with UV light (30 mJ). Proteins labeled with 4AzSA were detected by immunoblot (IB) analysis with α-SA antibodies. The membrane used for IB was stained with Coomassie Brilliant Blue (CBB) to assess consistency of loading. (FIG. 15B) MPK activation in wild-type (WT) Arabidopsis by AtHMGB3 and Pep1. Rep. #: independent replication number. For MPK activation experiments, Arabidopsis leaves were collected 15 min after infiltration (unless indicated otherwise) with 1 μM AtHMGB3 (or Pep1) and with the indicated concentrations of SA. Upper and lower panels are the immunoblot using α-pTEpY antibody and the CBB-stained membrane, respectively. Ctrl.: untreated, non-infiltrated control. Mock: infiltrated with same volume of 10 mM MgCl₂ into which the protein were diluted. (FIG. 15C) Effect of bak1-5, bak1-5/bkk1-1, and etr1 mutants on MPK activation by AtHMGB3 and Pep1. 1: Ctrl., 2: 1 μM AtHMGB3, 3: 1 μM Pep1. The ethylene receptor mutant etr1 served as another control. (FIG. 15D) Specific inhibition of AtHMGB3-, but not of Pep1-, induced MPK activation by SA. (FIG. 15E) AtHMGB3- and Pep1-induced callose deposition. For callose deposition experiments, Arabidopsis leaves were stained with aniline blue 15 h after infiltration with 100 nM AtHMGB3 or Pep1l. The data represent the mean±SD (n=20). Means that are statistically different (Tukey HSD test, P<0.05) are indicated by different letters over the bar. Means that are not statistically different are indicated by the same letter. (FIG. 15F) Compromised AtHMGB3-induced callose deposition in the bak1-5/bkk1-1 mutant. (FIG. 15G) Specific inhibition by SA of AtHMGB3-induced callose deposition, but not of Pep1-induced callose deposition. Mutant AtHMGB3 (R50A/K54A) has impaired SA inhibition of (FIG. 15H) MPK activation and (FIG. 15I) induction of callose deposition. All the above experiments were repeated more than two times with similar results.

FIG. 16A-FIG. 16G. SA binding by AtHMGB3 and inhibition of its DAMP activity. (FIG. 16A) Arabidopsis HMGBs bound 3AESA immobilized on a SPR sensor chip. (FIG. 16B) AtHMGB3- and Pep1-induced callose deposition. For callose deposition experiments, Arabidopsis leaves were stained with aniline blue 15 h after infiltration with 100 nM AtHMGB3 or Pep1. The data represent the mean±SD (n=20). Means that are statistically different (Tukey HSD test, P<0.05) are indicated by different letters over the bar. Means that are not statistically different are indicated by the same letter. (FIG. 16C) Compromised AtHMGB3-induced callose deposition in the bak1-5/bkk1-1 mutant. (FIG. 16D) Specific inhibition by SA of AtHMGB3-induced callose deposition, but not of Pep1-induced callose deposition. (FIG. 16E) Amino acid sequence alignment of HMG box domains of human and Arabidopsis HMGBs: AtHMGB1 (AT3G51880), AtHMGB2 (AT1G20693), AtHMGB3 (AT1G20696), AtHMGB4 (AT2G17560), AtHMGB5 (AT4G35570), AtHMGB6 (AT5G23420), AtHMGB12 (AT5G23405) and AtHMGB14 (AT2G34450). Multiple sequence alignments were performed by using the Clustal Omega (Version 1.2.1) (Sievers et al., 2011). Conserved Arg (R) and Lys (K) residues critical for SA binding are denoted by red letters and asterisks. The consensus and similar amino acid sequences are highlighted by black and grey colors, respectively. Dashes indicate spaces introduced into the amino acid sequences to enable proper alignment. (FIG. 16F) Amino acid sequence logo (Crooks et al., 2004) shows the conservation of the amino acid sequences of HMG boxes in human and Arabidopsis HMGBs. The Arg and Lys residues critical for SA binding are denoted by red asterisks. (FIG. 16G) Inhibition by SA of induction of callose deposition by WT but not by mutant (R50A/K54A) AtHMGB3. The figures shown in (B), (C), (D) and (G) are representative images for FIGS. 7E, 7F, 7G and 7I, respectively. The mean±SD shown below the figures are the same data shown in FIG. 7. Scale bars=100 μm. Means that are statistically different (Tukey HSD test, P<0.05) are indicated by different letters. Means that are not statistically different are indicated by the same letter.

### DETAILED DESCRIPTION OF THE INVENTION

Salicylic acid (SA) and its derivatives have been used for millennia to reduce pain, fever, and inflammation. In addition, prophylactic use of acetylsalicylic acid, commonly known as aspirin, reduces the risk of heart attack, stroke, and certain cancers. Since aspirin is rapidly de-acetylated by esterases in human plasma, much of aspirin's bioactivity can be attributed to its primary metabolite, SA. Here we demonstrate that human high mobility group box 1 (HMGB1) is a novel SA-binding protein. Extracellular HMGB1 is a damage-associated molecular pattern molecule, and has alternative redox states. SA suppresses both the chemotactic activity of reduced HMGB1 and the induction of the expression of pro-inflammatory cytokines and of COX-2 by disulfide-containing HMGB1. SA's inhibition of HMGB1 bioactivities occurs at concentrations 10-50 fold below that required to inhibit the cyclooxygenase activity of COX-2, in mouse cells where COX-2 is genetically ablated (Ptgs2−/−). In addition, we identified natural and synthetic SA derivatives that are more effective than SA at inhibiting HMGB1 bioactivities. SA-binding sites were identified by NMR analyses in the HMG-box domains Box A and Box B. The SA binding site in HMGB1 was mutated; mutant HMGB1 retained the chemo-attractant activity but could no longer be inhibited by SA and its derivatives. Our data indicate that HMGB1 is a pharmacological target of SA, distinct from COX-2.

The following definitions are provided to facilitate an understanding of the present invention.

### I. Definitions

A “HMGB activity modulator” refers to an agent which enhances or inhibits human HMGB1 function, as well as those of Arabidopsis thaliana HMGB1 and HMGB3. Such functions include, without limitation, DNA binding, cytokine induction, binding of salicylic acid, induction of chemotaxis, induction of autophagy, angiogenesis, and remodelling and repair of injured tissues and plant defense response induction such as callose deposition. SA binds Box A of HMGB1 with an affinity of ˜10 mM. Ac3AESA and Amorfrutin B appear to bind Box A more tightly (K_{d}<˜10 mM). In a preferred embodiment, the inhibitors of the invention bind HMGBs, including Homo sapiens HMGB1 (HsHMGB1), AtHMGB1 and AtHMGB3 with an affinity of K_{d}<10 mM.

“Pharmaceutically acceptable” indicates approval by a regulatory agency of the Federal government or a state government. “Pharmaceutically acceptable” agents may be listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A cell-free assay is any assay which does not involve use of an intact living cell, regardless of origin.

An in vitro assay is any assay which involves use of an intact living cell outside the organism, regardless of origin. We also use the term in vitro to describe biochemical assays and NMR experiments done in the absence of an intact living cell.

A in vivo assay is any assay which involves use of a whole organism, regardless of origin.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, which is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The terms “percent similarity,” “percent identity” and “percent homology,” when referring to a particular sequence, are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “oligonucleotides” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein, is generally found between exons, and is “spliced out” during processing of the mRNA transcript. As used herein, the term “exon” refers to a nucleic acid sequence found in genomic DNA that is predicted and/or experimentally confirmed to contribute contiguous sequence to a mature (e.g., spliced) mRNA transcript and/or is translated into protein. As used herein, the phrase “splice variants” refers to RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule, which may encode different amino acid sequences. The term splice variant may also refer to the proteins encoded by the above RNA molecules. As used herein, the phrase “alternative splicing” includes all types of RNA processing that lead to expression of plural protein isoforms from a single gene. As such, the phrase “splice variant” embraces mRNAs transcribed from a given gene that, however processed, collectively encode plural protein isoforms. For example, and by way of illustration only, splice variants can include exon insertions, exon extensions, exon truncations, exon deletions, alternatives in the 5′ untranslated region and alternatives in the 3′ untranslated region.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

The terms “transfected” and “transfection” as used herein refer to methods of delivering exogenous DNA into a cell. These methods involve a variety of techniques, such as treating cells with high concentrations of salt, an electric field, liposomes, polycationic micelles, or detergent, to render a host cell outer membrane or wall permeable to nucleic acid molecules of interest. These specified methods are not limiting and the invention relates to any transformation technique well known to a person of ordinary skill in the art.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997).

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The term “HSQC”, “HMQC”, and “TROSY-HSQC” refer to 2D NMR experiments which correlate interactions between ¹⁵N or ¹³C nuclei and covalently bound ¹H nuclei as described in Protein NMR Spectroscopy: Principles and Practice [Kindle Edition] John Cavanagh, Wayne J. Fairbrother, III, Arthur G. Palmer III, Nicholas J. Skelton, Mark Rance. 2ed Ed. 2007 Elsevier Inc.

### Screening Methods for Identifying Modulators of the Activities of HMGBs for Development of Therapeutic Agents

The methods described herein include methods (also referred to herein as “screening assays”) for identifying compounds that modulate (i.e., increase or decrease) activity of selected target protein (e.g., human HMGB1). Such compounds include, e.g., polypeptides, peptides, antibodies, peptidomimetics, peptoids, small inorganic molecules, small non-nucleic acid organic molecules, nucleic acids (e.g., anti-sense nucleic acids, siRNA, oligonucleotides, synthetic oligonucleotides), carbohydrates, or other agents that bind to the target proteins, have a stimulatory or inhibitory effect on, for example, expression of a target gene or activity of a target protein. Compounds thus identified can be used to modulate the activity of a target protein in a therapeutic protocol.

In general, screening assays involve assaying the effect of a test agent or compound on activity of a target protein in a test sample (i.e., a sample containing the target protein). Activity in the presence of the test compound or agent can be compared to activity in a control sample (i.e., a sample containing the target protein that is incubated under the same conditions, but without the test compound). A change in the activity of the target protein in the test sample compared to the control indicates that the test agent or compound modulates activity of the target protein and is a candidate agent.

Compounds can be tested for their ability to modulate one or more activities mediated by a HMGB1 protein as described herein. For example, compounds can be tested for their ability to modulate HMGB1 protein's binding to SA or SA derivatives such as 4-azidoSA (4AzSA) or 3-aminoethyl SA (3AESA). Methods of assaying a compound for such activities are known in the art. In some cases, a compound is tested for its ability to directly affect binding of SA or SA derivatives 4AzSA or 3AESA to HMGB1 proteins or alternatively tested for its ability to modulate a metabolic effect associated with the target protein, such as i) human HMGB1's induction of cytokine and Cox-2 gene expression, ii) human HMGB1's chemotaxis activity, iii) binding of human HMGB1 to CXCL12, and iv) plant HMGBs' induction of callose deposition.

In one embodiment, assays are provided for screening candidate or test compounds or agent that bind to a target protein or modulate the activity of a target protein in a manner comparable to SA. Such compounds include those that disrupt the interaction between HMGB1 and SA or its derivatives 4AzSA or 3AESA in assay such as, but not limited to, exclusion chromatography with radiolabelled SA (Chen et al., PNAS USA 90:9533-9537, 1993), photoaffinity crosslinking with 4AzSA (Tian etal., 2012), or surface plasma resonance (SPR) using 3AESA (Tian etal., 2012).

The test compounds used in the methods can be obtained using any of the numerous approaches in the art including combinatorial library methods, including: biological libraries; peptoid libraries (libraries of molecules having the functionalities of peptides, but with a novel, non-peptide backbone which are resistant to enzymatic degradation but which nevertheless remain bioactive; e.g., Zuckermann et al. (1994); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997).

Examples of methods for the synthesis of molecular libraries can be found in the literature, for example in: Cho et al., 1993; DeWitt et al, 1993, Carell et al., 1994, Erb et al., 1994, Gallop et al., 1994; and Zuckerman et al., 1994. Libraries of compounds may be presented in solution (e.g., Houghten, Bio/Techniques, 13:412421, 1992), or on beads (Lam, 1991), chips (Fodor, 1993), bacteria (U.S. Pat. No.5,223,409), spores (U.S. Pat. Nos. 5,571,698; 5,403,484; and 5,223,409), plasmids (Cull et al., 1992) or phage (Cwirla et al, 1990; Devlin et al, 1990; Scott and Smith, 1990, Felici et al., 1991).

In one embodiment, a cell-based assay is employed in which cells are exposed to or treated with a target protein in the presence or absence of a test compound. The ability of the test compound to modulate an activity of the target protein is then determined. The cell, for example, can be a cell of mammalian origin, e.g., rat, mouse, or human, a plant cell or a yeast cell.

In another embodiment, a whole organism-based assay is employed in which an organism is exposed to or treated with a target protein in the presence or absence of a test compound. The ability of the test compound to modulate an activity of the target protein is then determined. The organism, for example, can be mammalian e.g., rat, mouse, or human, a plant or a yeast.

In another aspect, the methods described herein pertain to a combination of two or more of the assays described herein. For example, a modulating agent can be identified using a a cell-free assay, and the ability of the agent to modulate the activity of a target protein can be confirmed in vivo, e.g., in an animal cell culture or whole animal or in plant cell culture or whole plant.

This invention further pertains to novel agents identified by the above-described screening assays. Accordingly, it is within the scope of this invention to further use an agent (compound) identified as described herein (e.g., a target protein modulating agent,) in an appropriate animal or plant models to determine the efficacy, toxicity, side effects, or mechanism of action, of treatment with such an agent. Furthermore, novel agents identified by the above-described screening assays can be used for treatments as described herein.

Compounds that modulate target protein activity (target protein modulators) can be tested for their ability to alter metabolic effects associated with the target HMGB 1 protein, e.g., with decreased expression or activity of target protein using methods known in the art and methods described herein. For example, the ability of a compound to modulate HMGB1 activities can be tested using an in vitro or in vivo model for disorders associated with aberrant HMGB1 function.

NMR for Ligand Screening. NMR chemical shift perturbations, of either the protein or the ligand, can be used for screening for small molecule lead compounds, validating initial small molecule hits identified with high-throughput or virtual screening assays, locating the corresponding binding site in the 3D protein structure, and to guide rational lead optimization. NMR methods uniquely complement crystallography studies. Strengths of NMR analysis methods include (i) the ability to detect even weak (K_{d} up to 1-10 mM) binding interactions, (ii) compatibility with systems that undergo structural changes upon ligand binding which may not be accommodated by a crystalline lattice, (iii) the ability to adjust solvent conditions over a wide range, and (iv) high sensitivity and very low sample requirements. Methods for studying protein-ligand interactions by NMR include methods that detect the ligand interaction by changes in the chemical shifts of the protein itself, referred to “protein-detected NMR experiments”, and methods that detect the ligand interaction by chemical shift changes of the ligand and/or magnetization transfer from the protein to the ligand, referred to a “ligand-detected NMR experiments” (Lepre, J. M, et al., 2004). Commonly used protein-detected NMR experiments include 1D-¹H-NMR, 1D-¹³C-NMR, and well as 2D ¹H-¹⁵N and 2D ¹H-¹³C HSQC, HMQC, TROSY-HSQC experiments which are well known to practitioners of the art. Commonly used ligand-detected NMR experiments include, but are not limited to,1D ¹H NMR experiments, water-LOGSY (Dalvit, C., et al., 2001) and saturation transfer difference (STD) (Meyer, B., and Peters, T (2003).

NMR spectroscopy techniques for screening and identifying ligand binding to protein receptors, Angewandte Chemie (International ed 42, 864-890) can be used to detect interactions between HMGB1 and small molecules. In the water-LOGSY (water-ligand observed via gradient spectroscopy) approach, bulk water magnetization is transferred to the ligand via the protein:ligand complex. These experiments can be used to screen intermediate sized (100-1000) compound libraries, and can detect even weak (e.g. 1-10 mM) protein-ligand interactions.

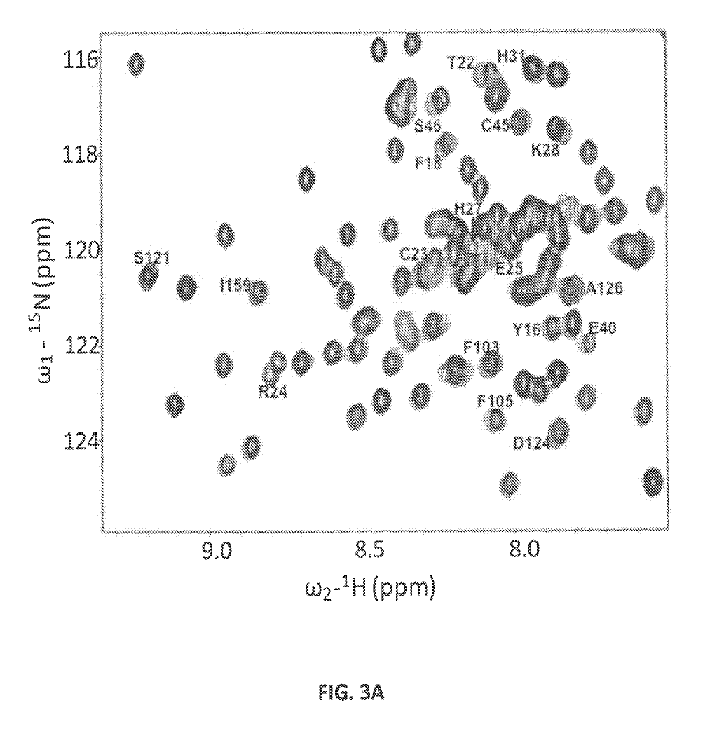

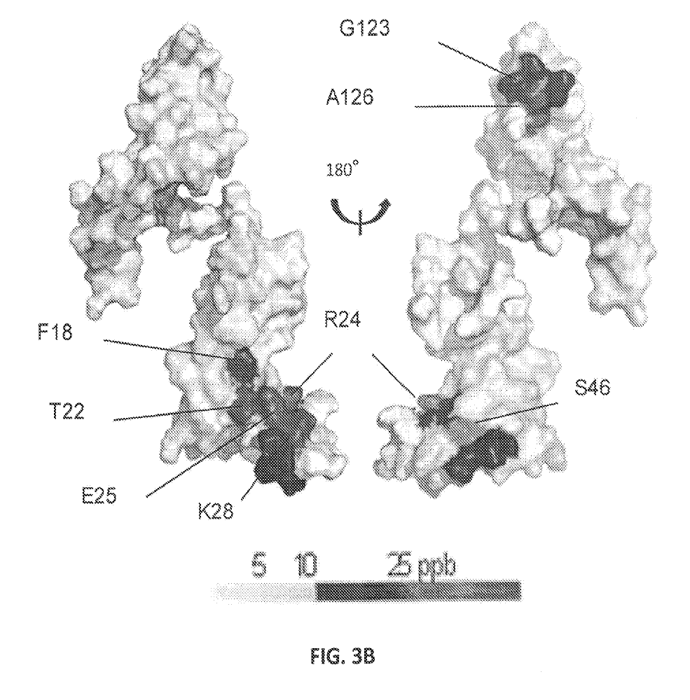

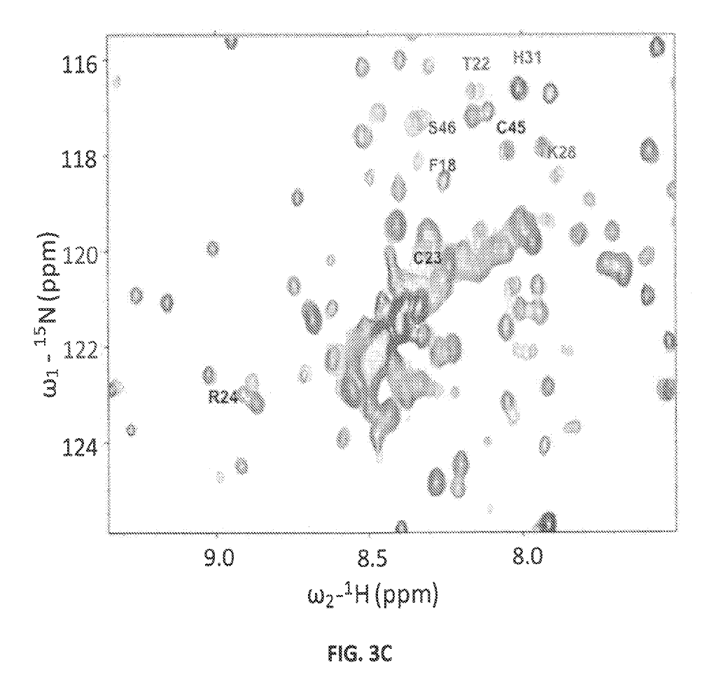

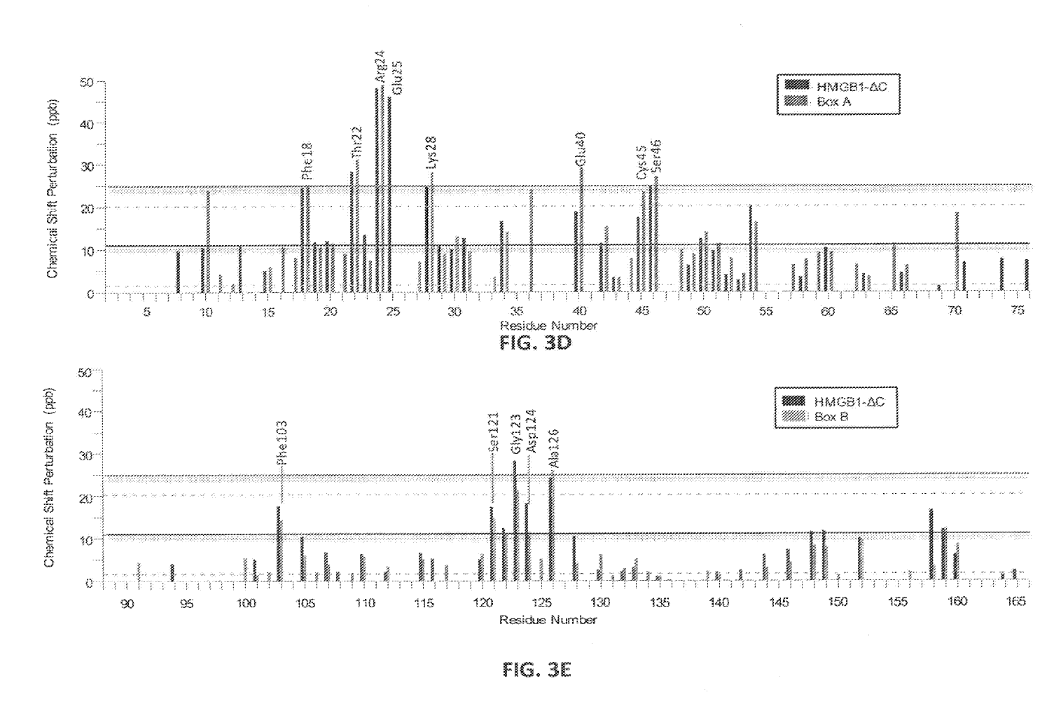

Using HMGB1 domain constructs that have already been shown to provide high quality HSQC spectra (e.g., FIG. 3), NMR chemical shift perturbation can be used to validate initial small molecule hits identified with high-throughput (htp) or virtual screening assays, including but not limited to fluorescence-based binding assays, locating the corresponding binding site in the 3D protein structure, and for rational lead optimization. Hits identified in htp screening assays will be characterized by assessing perturbations in HSQC spectra for which resonance assignments will be available, and interpreting these effects on the available 3D structures of HMGB1 effector domains. Some methods suitable for lead compound screening, identification, and optimization envisioned for use with our invention are summarized in C. A. Lepre, et al., (2004), which is incorporated herein by reference. The cryoprobes enable recording of high-quality HSQC data using 150 microliter samples at protein concentrations as low as 10 micromolar, and the 1.7 mm NMR probe provides high quality HSQC data on 35 microliter samples at 10 micromolar concentrations. Such NMR technologies allow NMR screening for lead compound optimization with as little as ˜500 nanomoles of each sample. Where appropriate, the 3D structures of tightly bound small molecule lead compounds may also be determined by NMR methods if they cannot be solved by crystallographic methods. These data will be used in redesign and lead optimization as outlined for crystallographic data above.

Crystallography for lead optimization. X-ray analysis has historically been very important to structure-based inhibitor design. Compounds identified as inhibitor “hits” by kinetic assays can be used to form complexes with the target protein. X-ray analysis will reveal the mode of inhibitor binding and act as a basis for further inhibitor design. Crystal•ligand complexes can also be formed by soaking inhibitors into preformed crystals, or complexes may be grown by co-crystallization. The latter case may be necessary if the complex triggers conformational changes. In either case, diffraction data is collected on a rotating anode source, processed, and converted to electron density maps. The orientation and occupancy of the inhibitors will be observed and compared with binding modes predicted from virtual screening or models from earlier design cycles. In this way, inhibitors analyzed in one cycle of design will feed into subsequent rounds of structural analysis and re-design in the general process of structure-based drug design (Kuhn 2002; Scapin et al., 2006).

Virtual Screening for SA Mimicks. In another aspect of the present invention, the 3D structures of HMGB1, SA-binding inhibitor molecules, SA analogs,and complexes between these are used for virtual (in silico) screening of compound libraries to identify additional lead compounds. These are further optimized using conventional structure activity relationships (SAR) approaches and designs based on the predicted and experimental structures of the complexes. As used herein, the term “virtual screening” refers to in silico design, using computer software to screen for possible binding ligands from a library of virtual molecules, allowing the characterization and refinement of small molecule inhibitors that will meet the design criteria for modifying the interactions between or the activity of HMGB1 based fully and/or at least in part on the published three dimensional (3D) coordinates together with the identification of the SA-binding residues in the structure based on NMR studies described herein.

The materials and methods set forth below were used to generate the data described herein below.

Identification of SA-Binding Protein from HeLa Cells

Approximately 3.5×10⁷ human HeLa cells were tryspinized and pelleted after neutralization, and resuspended in 2 mL of 0.2 M Tris-HCl (pH 7.4) containing 137 mM NaCl, 1 mM EDTA, 0.5% (v/v) Triton X-100, 1 mM PMSF and a protease inhibitor cocktail (Sigma). The suspension was then subjected to two freeze-thaw cycles, and cells were disrupted by ultrasonic sound. The solution was clarified by a 10 mM spin at 20,000 g, and dialyzed against loading buffer, 50 mM KPO₄ (pH 7.0) containing 50 mM NaCl, a protease inhibitor cocktail (Sigma) and 0.1% (v/v) Triton X-100.

SA-immobilized resin was prepared using a PharmaLink Immobilization Kit (Pierce), according to the manufacturer's instructions; the coupling with 0.5-1 mg SA typically resulted in ˜180 μg SA immobilized per mL resin. Following protein extract loading, the column was washed with loading buffer without and then with 0.1-10 mM 4-HBA to remove non-specifically bound proteins. Column-retained proteins were eluted with loading buffer containing 5 mM SA, and analyzed by SDS-PAGE. Three enriched proteins from the SA eluate were excised and subjected to MS (ESI-MS/MS) analyses for peptidic identifications (Donald Danforth Plant Science Center, St Louis, Mo.).

### Cloning, Expression, and Purification of HMGB1

The rat HMGB1 expression plasmid pET30 Xa/LIC HMGB1 was kindly provided by Dr. Lippard (Silver et al., 1995). Since there are two amino acid differences between rat and human HMGB1, we generated a human HMGB1 expression clone, pET30 Xa/LIC hHMGB1 by introducing PCR-based point mutations that converted residues 189D and 202E to 189E and 202D. Box A (residues 10-80) and Box B (residues 96-164) domains were amplified from pET30 Xa/LIC HMGB1 using suitable primers and cloned into Kpn1 and EcoRI sites of pET30 Xa/LIC.

Recombinant human HMGB1, Box A and Box B proteins were expressed in Escherichia coli strain BL21 cells grown at 37° C. to OD₆₀₀=0.7. HMGB1 expression was induced by adding 0.1 mM isopropyl-β-D-thiogalactopyrandoside (IPTG) for 16 h at room temperature. Cells were collected by centrifugation at 6,000 g for 30 min, and stored at −20° C. For protein purification, cells were resuspended in lysis buffer, buffer A (20 mM Tris-HC1/8.0, 0.15 M NaCl, 2 mM β-mercaptoethanol and 10% glycerol) containing 0.2% NP-40, 1 mg/ml lysozyme, 1 mM phenylmethyl sulphonyl fluoride and 10 mM imidazole. After sonication and centrifugation at 50,000 g for 1 h, soluble 6× His-tagged HMGB1 was purified by affinity chromatography using Ni-NTA agarose resin (Novagen); following washing the HMGB1-bound resin with buffer A containing 20 mM imidazole, HMGB1 was eluted in buffer A containing 300 mM imidazole. The 6× His tag was removed by overnight incubation at 4° C. with factor Xa (for full length HMGB1) and thrombin (for Box A or Box B). The cleaved HMGB1 was further purified using gel filtration chromatography on a HiLoad 16/60 Superdex 200 column (GE Healthcare) equilibrated in buffer A.

To prepare HMGB1^{RE}, purified HMGB1 was incubated with 5 mM DTT for 1 h at 4° C., and then desalted using a PD-10 desalting column (GE Healthcare) equilibrated in 20 mM Tris-HCl/8.0 and 20 mM NaCl plus 0.5 mM DTT. HMGB1^{SS} was prepared in the absence of any reducing agent. Contaminating LPS were removed from all HMGB1 preparations used for biological assays as described (Venereau et al., 2012).

For some experiments, HMGB1^{RE} and HMGB1^{SS} were provided by HMGBiotech (Milan, Italy).

### SA-Binding Analyses

Photoaffinity labeling and surface plasmon resonance (SPR) assays were performed essentially as described (Tian et al., 2012). For photoaffinity labeling, purified proteins (0.1 mg/ml) were incubated with 0.1 mM 4-azido salicylic acid (4-AzSA) in 100 μl 1×PBS buffer containing various concentrations of SA for 1 h on ice, followed by irradiation with 254 nm UV light at an energy level of 50 mJ, using a GS GENE Linker™ UV chamber (Bio-Rad). Aliquots of the reaction mixture were subjected to SDS-PAGE, transferred to a PVDF membrane, and detected by immunoblot analyses using an α-SA antibody (Acris). For SPR, 3-AESA was immobilized on a CM5 sensor chip (GE Healthcare) using the amine coupling kit (GE Healthcare). To test SA binding activity, 0.5 μM HMGB1 in HBS-EP buffer (0.01 M HEPES, pH 7.4, 0.15 M NaCl, 3 mM EDTA, 0.005% v/v Surfactant P20; GE Healthcare) without or with varying concentrations of SA was flowed through 3-AESA-immobilized or mock-coupled flow cells. The binding signals were determined by subtracting out the signal from the mock-coupled flow cell.

### NMR Sample Preparation

All four HMGB1 constructs (Box A, Box B, HMGB1-ΔC, and full length HMGB1) were cloned in either pET15Tev_NESG or Avi-NESG expression vectors, which both include a TEV-cleavable N-terminal hexaHis purification tag, expressed with uniform ¹⁵N or ¹³C,¹⁵N-enrichment, and purified as previously described (Xiao et al., 2010; Acton, T. B.; Xiao, R.; Anderson, S.; Aramini, J. M.; Buchwald, W.; Ciccosanti, C.; Conover, K.; Everett, J. K.; Hamilton, K.; Huang, Y. J.; Janjua, H.; Kornhaber, G. J.; Lau, J.; Lee, D. Y.; Liu, G.; Maglaqui, M.; Ma, L.-C.; Mao, L.; Patel, D.; Rossi, P.; Sandev, S.; Sharma, S.; Shastry, R.; Swapna, G. V. T.; Tang, Y.; Tong, S. N.; Wang, D.; Wang, H.; Zhao, L.; Montelione, G. T. Meth. Enzymology 2011, 493: 21-60). Protein constructs were purified with a 5 mL HisTrap HP NiNTA column (GE Healthcare), cleaved with TEV-protease, and repurified using a 5 mL HisTrap HP column. Size exclusion chromatography was performed using a HiLoad 26/600 Superdex 75 column (GE Healthcare) with a buffer containing 50 mM Tris-HCl, pH 7.5, 100 mM NaCl, 0.02% NaN₃, and 10 mM DTT. Samples for NMR studies were exchanged into this same buffer, containing either no SA or 10 mM SA, using a Zeba 96-well spin-desalting plate (Thermo Scientific).

### NMR Data Collection

NMR samples (50-200 μL) contained 0.25 mM protein in 20 mM NaPO₄, pH 7.5, 100 mM NaCl, 0.02% NaN₃, 1 mM TCEP. Samples were analyzed with and without SA or other candidate inhibitors at 25° C. Chemical shift perturbation studies were carried out using either a Bruker Avance 800 MHz Avance NMR system with 4-mm NMR tubes, or using a Bruker Avance 600 MHz NMR spectrometer equipped with a 1.7 mm micro cryoprobe. 1024 or 2048 complex points were acquired for each of 80 to 200 increments in the ¹⁵N dimension with a recycle delay of 1 s. Each spectrum was typically acquired for 2-8 hrs. Chemical shift assignments were determined using a uniformly ¹³C,¹⁵N-enriched HMGB1-ΔC construct at 25° C. via traditional triple-resonance NMR experiments (Biamonti et al., 1994; Moseley et al., 2001) recorded on a Bruker 800 MHz NMR spectrometer equipped with a 5 mm cryoprobe. Disulfide oxidation studies were carried out using the ¹⁵N-¹H SOFAST-HMQC NMR experiment (Schanda & Brutscher, 2005; Schanda et al., 2005; Zandarashvili et al., 2013).

### Cells

Human HeLa cells were grown in T75 flasks in DMEM medium supplemented with penicillin (10,000 IU/mL) streptomycin (10 mg/μL), and 10% fetal calf serum. Ptgs2−/− MEFs were derived from Ptgs2−/− E16 mouse embryos, obtained from the mating of Ptgs2+/− mice purchased from Taconic Biosciences. PBMCs were isolated from buffy coats of donor blood (Hospital of Magenta, Milan, Italy) by Ficoll gradient centrifugation (Lymphoprep; AXIS-SHIELD. CD14+ monocytes were isolated by positive immunoselection (CD14 Microbeads; Miltenyi Biotec) according to the manufacturer's instructions, and differentiated into macrophages using X-Vivo medium supplemented with 1% heat inactivated human serum, GM-CSF, and M-CSF.

### Assays for Cytokine Induction

Macrophages were exposed to 3 μg/ml HMGB1^{SS} or 50 ng/ml LPS. Total RNAs were isolated using the Illustra RNAspin Mini kit (GE Healthcare), and complementary DNA (cDNA) was obtained by retro-transcription with oligo(dT) primers (Invitrogen, Carlsbad, Calif., USA) and SuperScript II Reverse Transcriptase (Invitrogen) following the manufacturers' instructions. Quantitative real-time PCR was performed using a LightCycler480 (Roche Molecular Diagnostics), in duplicates, using SYBR Green I master mix and the oligonucleotides listed in Table of primers below. The ΔCt method was used for quantification, and the β-actin gene was used for normalization.

### Chemotaxis Assay

Cell migration was assayed using Boyden chambers (Venereau et al., 2012). The downside of PVP-free polycarbonate filters (8 μm pores; Millipore) was coated with 50 μg/ml fibronectin (Sigma). Serum-free DMEM (negative control), DMEM containing 0.1 nM fMLP (positive control; Sigma), or DMEM containing 1 nM HMGB1^{RE} or HMGB1^{SS} were placed in the lower chamber, together with different concentrations of SA (Sigma). The mouse 3T3 fibroblast cells were cultured in DMEM media supplemented with 10% CS until 90% confluence, washed twice with PBS to eliminate any floating cells, and harvested with trypsin. 50,000 cells in 200 μL were added to the upper chamber, and cells were left to migrate for 3 h at 37° C. Non-migrating cells were removed with a cotton swab, and migrated cells were fixed with ethanol and stained with Giemsa.

Since there are two amino acid differences between rat and human HMGB1, we generated a human HMGB1 expression clone, pET30 Xa/LIC hHMGB1, from pET30 Xa/LIC ratHMGB1 by introducing PCR-based point mutations that converted residues 189D and 202E to 189E and 202D (Silver et al., 1995). Box A (residues 10-80) and Box B (residues 96-164) domains were amplified from pET30 Xa/LIC HMGB1 and cloned into KpnI and EcoRI sites of pET30 Xa/LIC. The AtHMGB1 and AtHMGB3 clones were purchased from The Arabidopsis Information Resource (TAIR; ABRC stock number: DKLAT3G51880 and DKLAT1G20696), and the ORFs were amplified and cloned into KpnI and EcoRI sites of pET30 Xa/LIC.

Recombinant HMGB proteins, including human HMGB1, Box A and Box B, and Arabidopsis AtHMGB1 and AtHMGB3, were expressed in Escherichia coli strain BL21 cells grown at 37° C. to OD₆₀₀=0.7. Expression of HMGB encoding genes was induced by adding 0.1 mM isopropyl-βD-thiogalactopyrandoside (IPTG) for 16 h at room temperature. Cells were collected by centrifugation at 6,000 g for 30 min, and stored at −20° C. For protein purification, cells were resuspended in lysis buffer, buffer A (20 mM Tris-HCl/8.0, 0.15 M NaCl, 2 mM β-mercaptoethanol and 10% glycerol) containing 0.2% NP-40, 1 mg/ml lysozyme, 1 mM phenylmethyl sulphonyl fluoride (PMSF) and 10 mM imidazole. After sonication and centrifugation at 50,000 g for 1 h, soluble 6× His-tagged HMGB proteins were purified by affinity chromatography using Ni—NTA agarose resin (Novagen); following washing with buffer A containing 20 mM imidazole and 1.0% Triton X-100, HMGB proteins were eluted in buffer A containing 300 mM imidazole. HMGB proteins were further purified using gel filtration chromatography on a HiLoad 16/60 Superdex 200 column (GE Healthcare) equilibrated in buffer A.

To prepare HMGB1^{RE}, purified HMGB1 was incubated with 5 mM DTT for 1 h at 4° C., and then desalted using a PD-10 desalting column (GE Healthcare) equilibrated in 20 mM Tris-HCl/8.0 and 20 mM NaCl plus 0.5 mM DTT. HMGB1^{SS} was prepared in the absence of any reducing agent. Contaminating LPS were removed from all HMGB1 preparations used for biological assays as described (Venereau et al., 2012).

For some experiments, HMGB1^{RE} and HMGB1^{SS} were provided by HMGBiotech (Milan, Italy).

### MAPK Activation Assay

To detect the phosphorylation of Arabidopsis MAPKs (MPK3/4/6), 3 to 4 week-old soil grown Arabidopsis leaves were infiltrated with 1 μM AtHMGB3 or 1 μM Pep1 peptide (ATKVKAKQRGKEKVSSGRPGQHN; synthesized by GenScript USA Inc.). Two leaf disks were cut from the leaf center of two different leaves using a cork borer (diameter=0.7 cm) and immediately frozen and ground to fine powder in liquid nitrogen. Proteins were extracted using lacus buffer (25 mM Tris-HCl, pH 7.5, 15 mM MgCl₂, 15 mM EGTA, 75 mM NaCl, 1 mM DTT, 0.1% NP-40, 5 mM p-nitrophenylphosphate, 60 mM β-glycerophosphate, 0.1 mM Na₃VO₃, 1 mM NaF, 1 mM PMSF, 5 μg/ml leupeptin, 5 μg/ml aprotinin), and then centrifuged at 15,000 g for 10 min at 4 ° C. to remove cell debris. The protein concentration of supernatant was measured using the Bradford reagent (Bio-Rad). Forty μg of proteins per sample was separated on the 8% SDS-PAGE and transferred to a PVDF membrane for immunoblotting using the anti-phospho-p44/42 MAPK antibody (Cell Signaling Technology). The large subunit of Rubisco was visualized by Coomassie Brilliant Blue (CBB) staining of the PVDF membrane in order to assess consistency of loading.

### Callose Staining

Arabidopsis leaves were collected 15 h after infiltration with 100 nM AtHMGB3 or 100 nM Pep1, and then immediately immersed in alcoholic lactophenol (phenol:glycerol:lactic acid:water:ethanol=1:1:1:1:8) to destain the chlorophyll. To remove the background fluorescence, destained leaves were treated with toluidine blue O solution (0.05% toluidine blue O, 0.1 M sodium acetate, pH 4.4) for 30 min, and then rinsed twice with 150 mM K₂HPO₄ (pH 9.5). Callose was stained with aniline blue solution (0.02% aniline blue, 150 mM K₂HPO₄, pH 9.5) for 30 min in the absence of light. The aniline blue-stained leaves were mounted with 25% glycerol and analyzed using a Leica DM5500 epifluorescence microscope. The number of callose loci was quantified using an ImageJ (NIH).

### Statistical Analysis

The results are expressed as a mean±standard deviation (SD). Statistical analyses was done using a Tukey honest significant difference (HSD) test with JMP Pro, version 10.0.0, software (SAS Institute Inc.).

Primers Used in this Study.

| | Forward and Reverse Primer |
|---|---|
| Point mutation of HMGB1 | F1: 5′-AAATTAATACGACTCACTATAGGGG-3′ (1)* |
| (D189E and E202D) | F2: 5′-AGACGAGGAGGATGAAGAGGATGAGGAAGAGGAGGAAGATGAGGAAG-3′ |
| | (2) |
| | R1: 5′- ATCTTCCTCCTCTTCCTCATCCTCTTCATCCTCCTCGTCTTCTTCC-3′ (3) |
| | R2: 5′-ATCCGGATATAGTTCCTCCTTTC-3′ (4) |
| BoxA cloning | F: 5′-GGTACCATGGGCAAAGGAGATCCTAAG-3′ (KpnI) (5) |
| into pET30 Xa/LIC | R: 5′-GAATTCTTAGAACTTCTTTTTGGTCTCCCCTTTGG-3′ (EcoRI) (6) |
| BoxB cloning | F: 5′-GGTACCAAAAAGAAGTTCAAGGACCCCAATGC-3′ (KpnI) (7) |
| into pET30 Xa/LIC | R: 5′-GAATTCTTATTTAGCTCTGTAGGCAGCAATATCCTTC-3′ (EcoRI) (8) |
| EMSA Probe | F: 5′-CCCTCTAGA NNNNNNNNNNNNNNNNNNNNNNGAATTCGGGCC-3′ (9) |
| | R: 5′-GGCCCGAATTC-3′ (10) |
| β-actin | F: 5′-AGCCATGTACGTAGCCATCC-3′ (11) |
| | R: 5′-CTCTCAGCTGTGGTGGTGAA-3′ (12) |
| COX-2 | F: 5′-AGAAGGAAATGGCTGCAGAA-3′ (13) |
| | R: 5′-GCTCGGCTTCCAGTATTGAG-3′ (14) |
| Murine IL-6 | F: 5′-CTGGGAAATCGTGGAAATGAG-3′ (15) |
| | R: 5′-CTCTGGCTTTGTCTTTCTTGTTATC-3′ (16) |
| Murine II-1beta | F: 5′-GCTACCTGTGTCTTTCCCGTG-3′ (17) |
| | R: 5′-GGGTGTGCCGTCTTTCATTAC-3′ (18) |
| Murine COX-2 | F: 5′- ACCGTGGGGAATGTATGAGC-3′ (19) |
| | R: 5-GTTTGGGCAGTCATCTGCTACG-3′ (20) |
| Four-way junction DNA | A: 5′-GTCCAGCACGAGTCCTAACGCCAGGC-3′ (21) |
| | B: 5′-GGGCCTGGCGTTAGGTGATACCGATCGG-3′ (22) |
| | C: 5′-CCGATCGGTATCAGGCTTACGACGAG-3′ (23) |
| | D: 5′-GGCTCGTCGTAAGCCACTCGTGCTGGAC-3′ (24) |

*numbers in parentheses are SEQ ID NOS:

The following example is provided to illustrate certain embodiments of the invention. It is not intended to limit the invention in any way.

### EXAMPLE I

### Identification of HMGB1 as an SA-Binding Protein

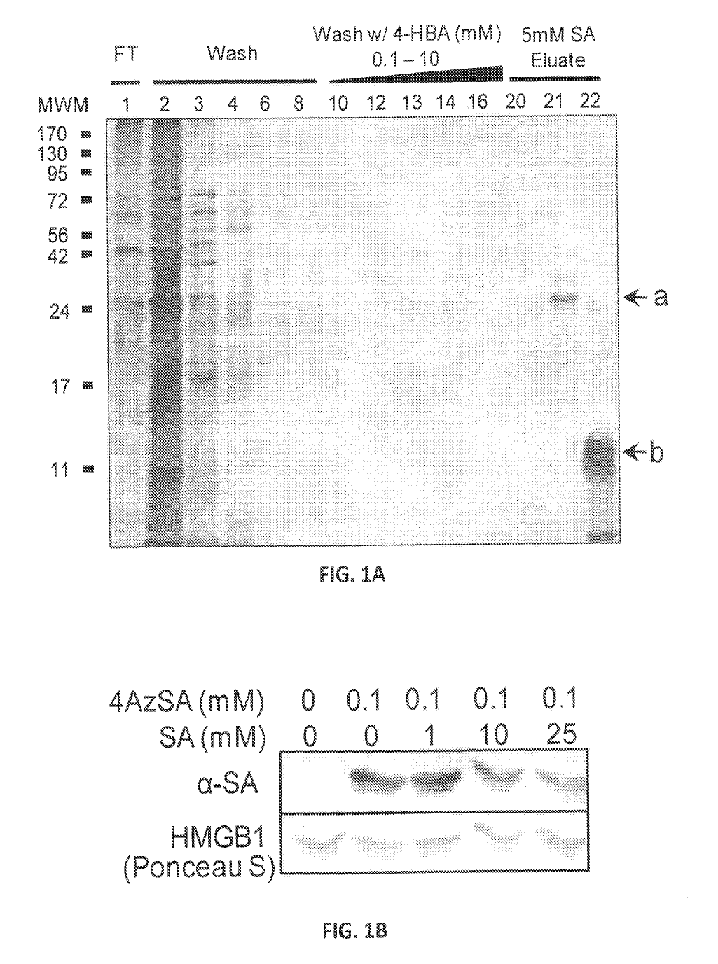

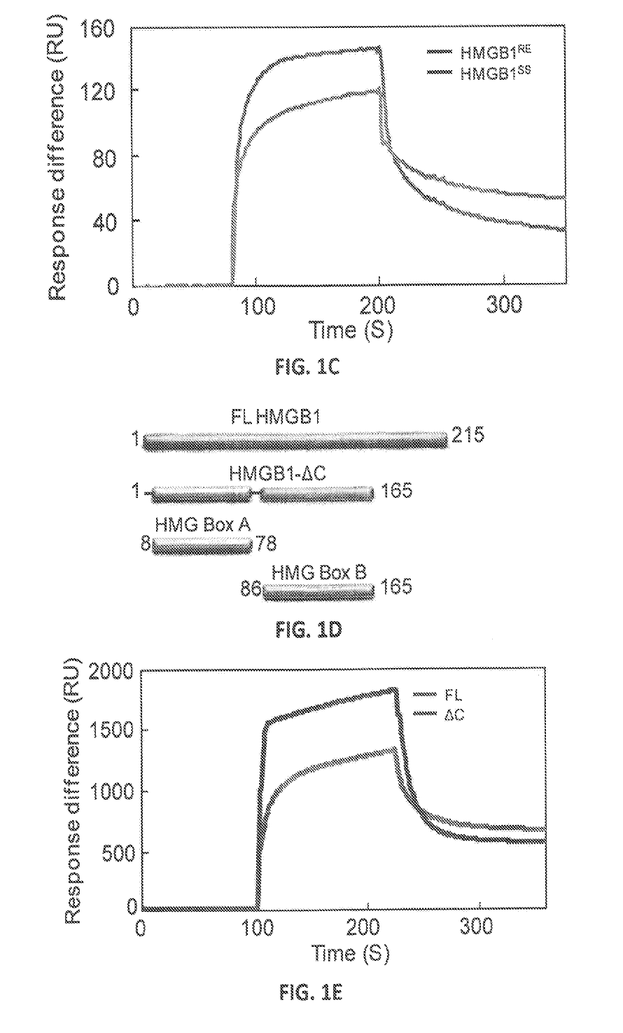

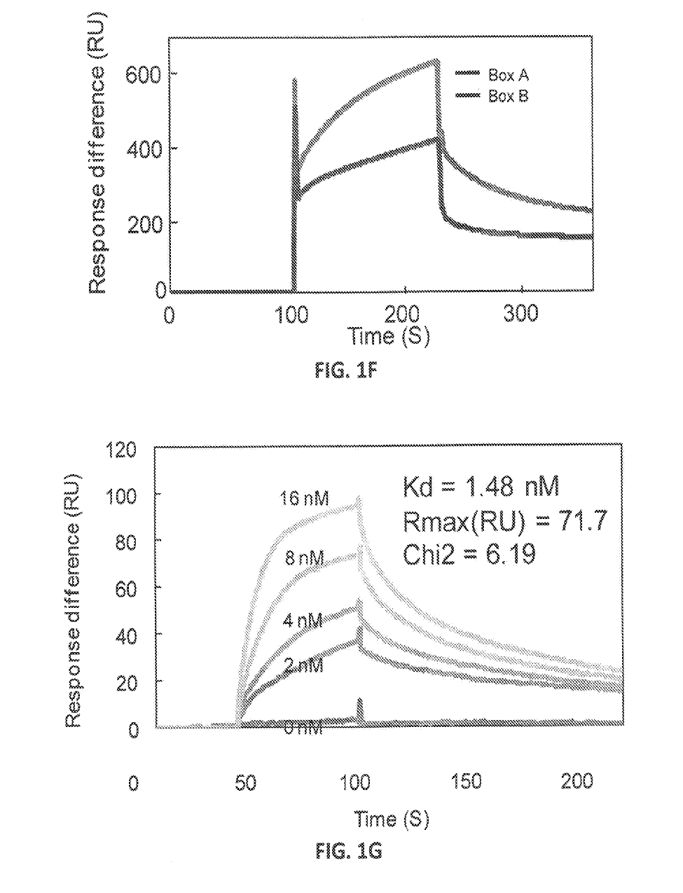

To identify novel salicylate effectors and/or receptors, we subjected HeLa cell extracts to affinity chromatography on a PharmaLink column to which SA had been linked (FIG. 1A). Proteins that non-specifically bound the SA-linked matrix were removed using the biologically inactive SA analog, 4-hydroxy benzoic acid (4-HBA) in a stringent washing step. The affinity column was then competitively eluted with a high concentration of SA and the released proteins were identified by mass spectroscopy. HMGB1 (GI:7669492) was repeatedly selected via this approach.

HMGB1's SA-binding activity was then assessed using photoaffinity-crosslinking. Recombinant HMGB1 was pre-incubated with or without photoreactive 4-azido SA (4AzSA) before exposure to UV irradiation (FIG. 1B). Immunoblots of reaction products probed with α-SA antibody revealed a band at the expected molecular weight for HMGB1 when 4AzSA was present in the reaction, but not in the control reaction without 4AzSA. Increasing concentrations of SA effectively inhibited crosslinking of 4AzSA to HMGB1, indicating that HMGB1's binding to this SA derivative reflects authentic SA-binding activity.

To further confirm HMGB1's SA-binding activity, its binding to the SA derivative 3-aminoethyl SA (3AESA) was analyzed using surface plasmon resonance (SPR). SPR provides highly sensitive and quantitative measurements of bimolecular interactions in real-time (Tian et al., 2012). To facilitate detection of binding, HMGB1 was flowed over a sensor chip onto which 3AESA was immobilized. Recombinant HMGB1 exhibited strong binding to the immobilized 3AESA (FIG. 1 C-G).

HMGB1 has multiple redox states, which in part depend on a reversible intra-molecular disulfide bond formed between cysteine residues 23 and 45 (Venereau et al., 2012). Both reduced and disulfide-bonded forms, hereafter referred to as HMGB1^{RE} and HMGB1^{SS}, bound 3AESA (FIG. 1C). To identify which domain(s) of HMGB1 binds SA, four constructs were generated: (i) full length: residues 2-215, (ii) HMGB1-ΔC: residues 2-166, (iii) Box A: residues 10-80, and (iv) Box B: residues 96-164 (FIG. 1D). SPR analysis revealed that all four constructs exhibited SA-binding activity, indicating that Box A and Box B each have an SA-binding site (FIG. 1, FIG. 1E-FIG. 1G).

### SA Inhibits HMGB1's Chemo-Attractant Activity

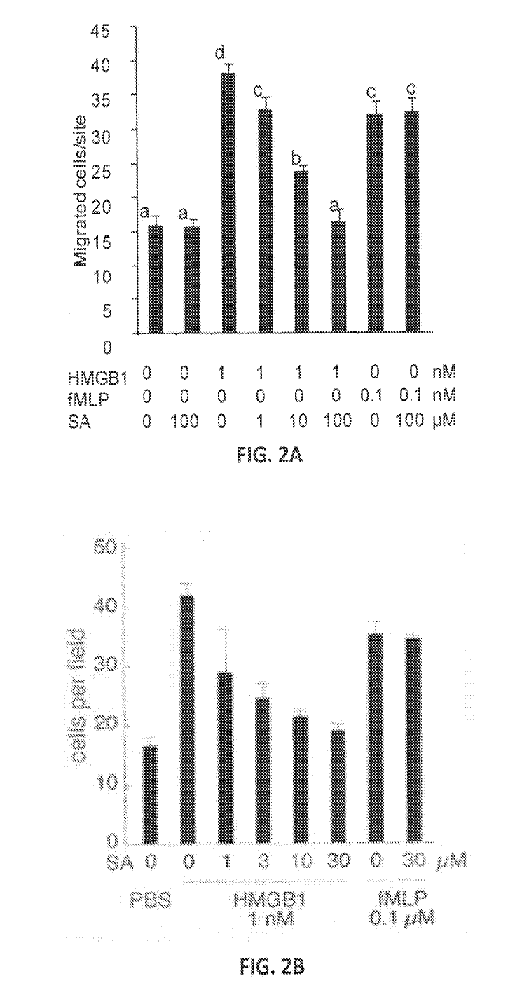

HMGB1^{RE} acts as a chemo-attractant that mediates migration of several cell types, including monocytes, fibroblasts, mesoangioblasts, smooth muscle cells, and colon cancer cells (Venereau et al., 2012). To determine whether SA alters HMGB 1's chemo-attractant activity, a chemotaxis assay was performed with mouse 3T3 fibroblasts. SA strongly inhibited HMGB1^{RE}-elicited migration of fibroblasts in a dose-dependent manner, with an IC₅₀ of approximately 3-4 μM (FIG. 2A). By contrast, SA did not affect fibroblast migration induced by the well-known chemo-attractant N-formyl-methionyl-leucyl-phenylalanine (fMLP). Thus, SA appears to specifically inhibit the chemo-attractant activity of HMGB1^{RE}, rather than suppress all cell migration per se.

The primary action of aspirin in mammals has been attributed to its effects on cyclooxygenases (COX) (Ekinci, 2011). COX-2 deficiency impairs cell adhesion and migration of macrophages (Díaz-Muñoz, Osma-García, Iñiguez, & Fresno, 2013). To test whether SA-mediated inhibition of chemotaxis was mediated via COX-2 rather than HMGB1, we performed the cell migration assay with mouse embryonic fibroblasts (MEFs), either wild type (WT) or genetically ablated for the gene coding for COX-2 (Pgts2−/−). SA effectively inhibited fibroblast migration in a dose-dependent manner, in both WT and Ptgs2−/− MEFs (FIG. 2B).

### Identification of the Sites of SA Binding in HMGB1

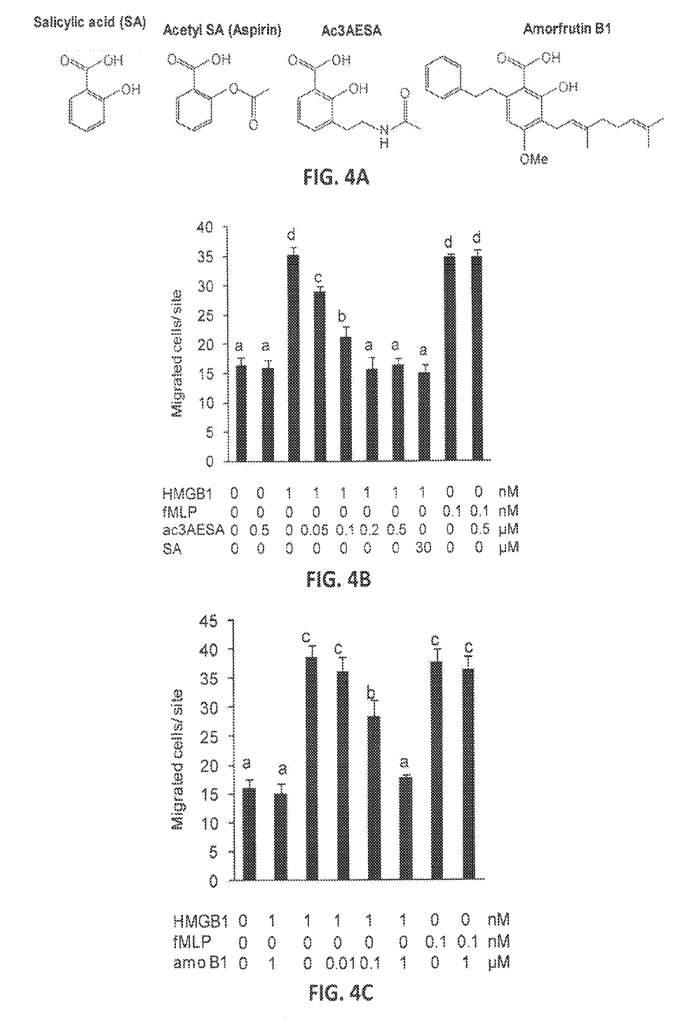

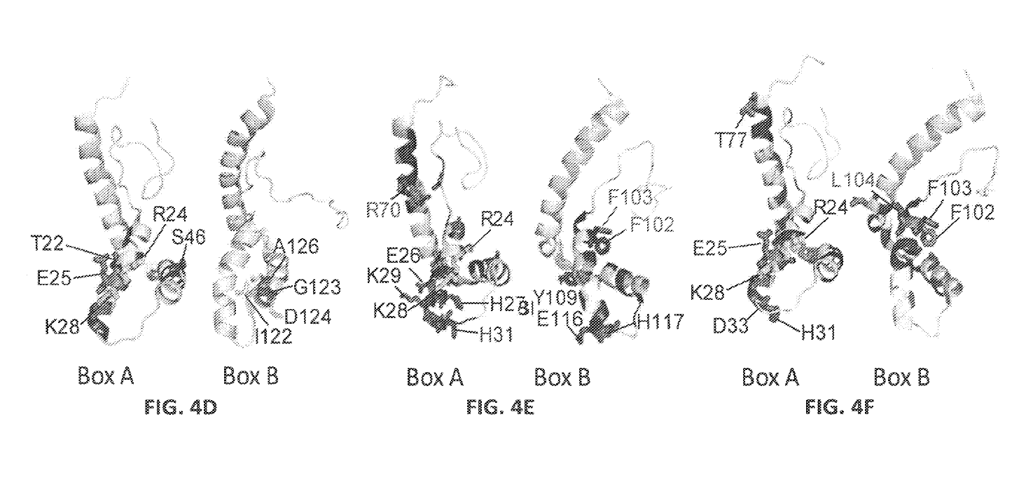

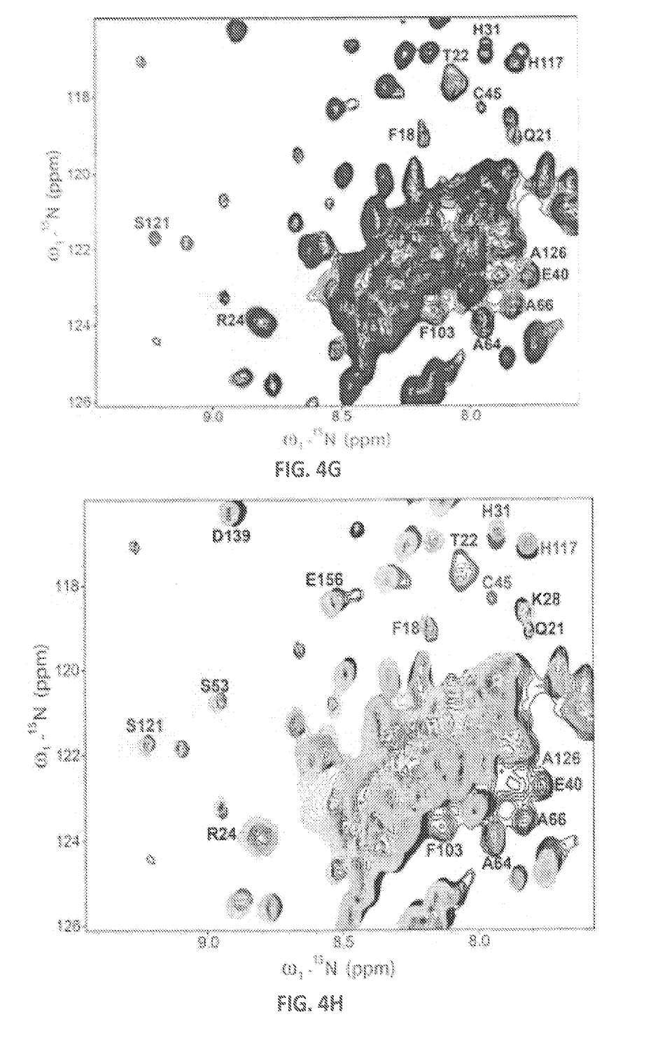

The SA-binding sites of HMGB1^{RE} were identified by ¹⁵N-¹H heteronuclear single quantum correlation (HSQC) 2D NMR spectroscopy at pH 7.5. Under these conditions, some surface amide protons are broadened due to solvent exchange, and cannot be observed. We determined the sequence-specific resonance assignments for all observable backbone amide ¹⁵N and ¹H^{N} resonances of HMGB1-ΔC using triple-resonance NMR methods. A region of the superimposed ¹⁵N-¹H-HSQC spectra obtained in the presence or absence of SA is shown in FIG. 3A. Analysis of¹⁵N-¹H HSQC spectra from HMGB1-ΔC showed that some backbone amide ¹H^{N} and/or N resonances exhibited significant chemical shift perturbations (CSPs) upon SA binding, including those of residues Phe18, Thr22, Arg24, Glu25, His27, Lys28, Glu40, Cys45, and Ser46 in Box A and Phe103, Ser121, Gly123, Asp124, and Ala126 in Box B. Similar results were obtained using full-length HMGB1 (FIG. 4G), HMGB1-ΔC (FIG. 3C, D, E) and using Box A or Box B alone (FIG. 3D, E), confirming that each domain has an SA-binding site. Models of HMGB1 reveal that the CSPs are localized in the corresponding regions of the two HMG-box domains.

### SA Derivatives, which Share the Same Binding Sites on HMGB1, are More Potent Inhibitors of its Chemo-Attractant Activity than SA

Dissociation constants derived by titrating SA with either Box A or full-length HMGB1 suggested a K_{d} in the range of 10 mM, which is scarcely compatible with a low micromolar IC₅₀ for chemo-attractant activity of HMGB1. In contrast, HMGB1^{RE} bond to 3AESA on the SPR sensor chip with much higher affinity with an apparent Kd of approximately 1-2 nM (FIG. 1G). Thus, HMGB1^{RE} appears to bind weakly to SA, but with higher affinity to both the PharmaLink column bearing crosslinked SA and to the 3AESA-bound SPR sensor chip. SA crosslinking to the PharmaLink column involves a Mannich reaction, which introduces an alkyl group onto the phenyl ring of SA. Likewise, 3AESA contains an alkyl group on the phenyl ring of SA. This suggested that SA derivatives containing alkyl groups might be better binders than SA. To test this possibility, a new SA derivative, acetyl-3-aminoethyl-SA (ac3AESA) was synthesized from 3AESA by converting the charged amino moiety to a neutral acetylated amide to resemble 3AESA linked to the SPR chip (FIG. 4A). In addition, we identified promising natural SA derivatives, the amorfrutins, from the medicinal legume licorice Glycyrrhiza foetida. It also contains glycyrrhizin, a known HMGB1 inhibitor (Mollica et al., 2007).

Amorfrutins recently have been shown to have potent anti-diabetic activity, which was ascribed to their ability to bind to and activate the nuclear receptor peroxisome proliferator-activated receptor gamma (Weidner et al., 2012). Amorfrutins contain a phenyl ring with free carbonyl and hydroxyl groups at positions 1 and 2, respectively. This is the SA core, which we have found is critical for biological activity of SA derivatives in plants. Amorfrutin B1 structure is compared to those of SA, aspirin, and ac3AESA in FIG. 4A.

Comparison of the NMR spectra of HMGB1 in the absence vs presence of 15 mM SA, 3 mM ac3AESA, or 3 mM amorfrutin B1 revealed that most of the same amino acid residues displayed significant CSPs in the presence of SA and its derivatives (FIG. 4 D-F). Mapping the ac3AESA- and amorfrutin B 1-induced CSPs onto the structure of human HMGB1 (2YRQ, residues 6-164) confirmed that SA, ac3AESA and amorfrutin B1 share the same binding sites on Box A and Box B (FIG. 4 D-F). More importantly, these two SA derivatives showed increased potency in suppressing HMGB1's chemo-attractant activity. Ac3AESA was 40-60 times (IC₅₀ of approximately 70 nM), while amorfrutin B1 was approximately 50-70 times (IC₅₀ of approximately 60 nM), more potent than SA at inhibiting the chemo-attractant activity of HMGB1^{RE}.

Ac3AESA Binds HMGB1 More Tightly than SA Itself.

NMR can be used to detect protein ligand interactions either by protein-detected experiments (Lepre, Moore et al. 2004), detecting changes in the frequencies of resonances of the protein sample due to ligand binding (protein-detected chemical shift perturbation (CSP) experiments), or by ligand-detected experiments (Lepre, Moore et al. 2004), in which NMR properties of the ligands due to ligand-protein interactions are detected. Protein-detected ligand binding experiments are usually done using isotope-enriched protein samples which can be studied by isotope-filtered NMR experiments, including for example ¹⁵N-¹H-HSQC (Kay, Keifer et al. 1992). Ligand-detected experiments include the Waterlogsy experiment (Meyer and Peters 2003, Lepre, Moore et al. 2004, Furihata, Shimotakahara et al. 2008), which detects interactions in the ternary complex formed by the protein, the ligand, and one or more water molecules, and the Saturation Transfer Difference (STD) NMR experiments (Mayer and Meyer 2001, Lepre, Moore et al. 2004, Wang, Liu et al. 2004), which detects magnetization transfer from the protein to the ligand in the protein-ligand complex. The Waterlogsy experiment is a more sensitive experiment, typically employed to detect qualitatively if there is an interaction between protein and ligand. The STD experiment is less sensitive, but more straight forward to interpret in terms of relative ligand binding affinities (Wang, Liu et al. 2004).

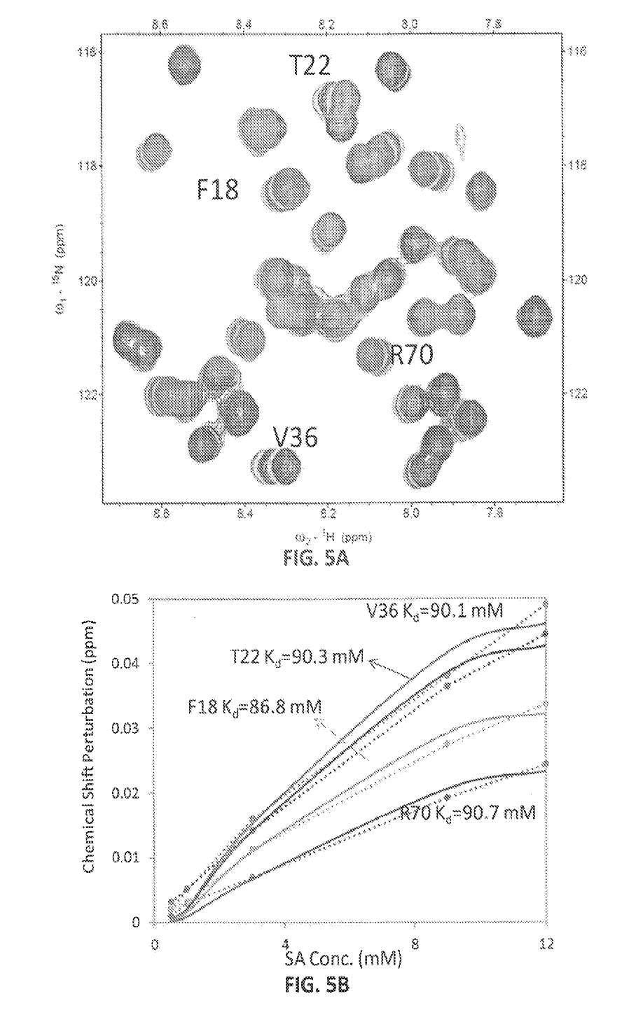

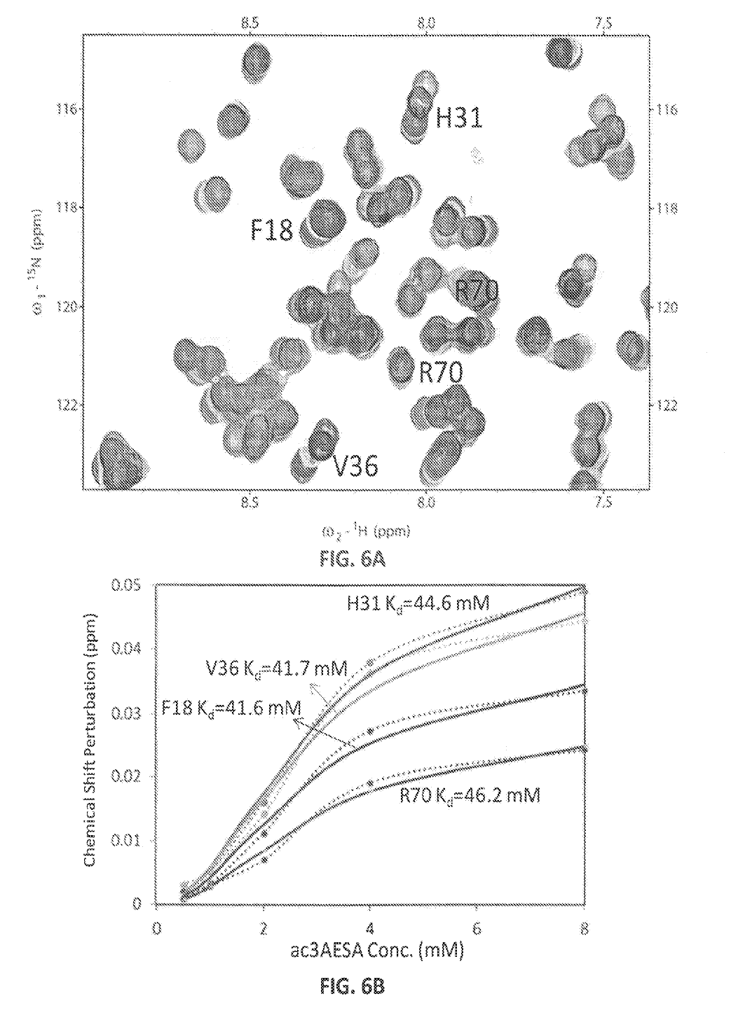

NMR experiments were used to discover that ac3AESA binds to HMGB1 Box A domain of HMGB1 more tightly than SA itself. FIGS. 5 and 6 provide protein-detected ¹⁵N-¹H HSQC chemical shift perturbation data demonstrating SA and ac3AESA binding to the Box A domain of HMGB1 in a buffer containing 20 mM Na₂H(PO₄), 100 mM NaCl, 0.02% NaN₃, 1 mM TECP, pH 7.5. Chemical shift perturbations (CSPs) are defined as Δδₚₚₘ=[(δ_{HN}*6.5)²+δ_{N}²]^{1/2}. Titration data shown in panels B of FIGS. 5 and 6 demonstrate that ac3AESA binds more tightly to HMGB1 Box A (K_{d}˜45 mM) than SA itself (K_{d}˜90 mM) under the conditions of these experiments.

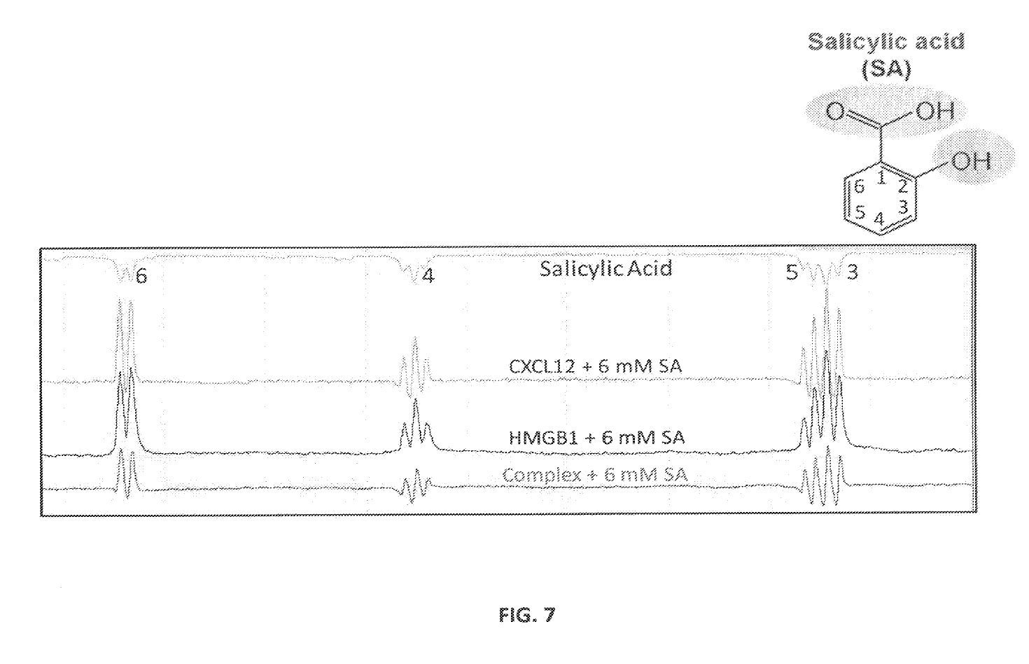

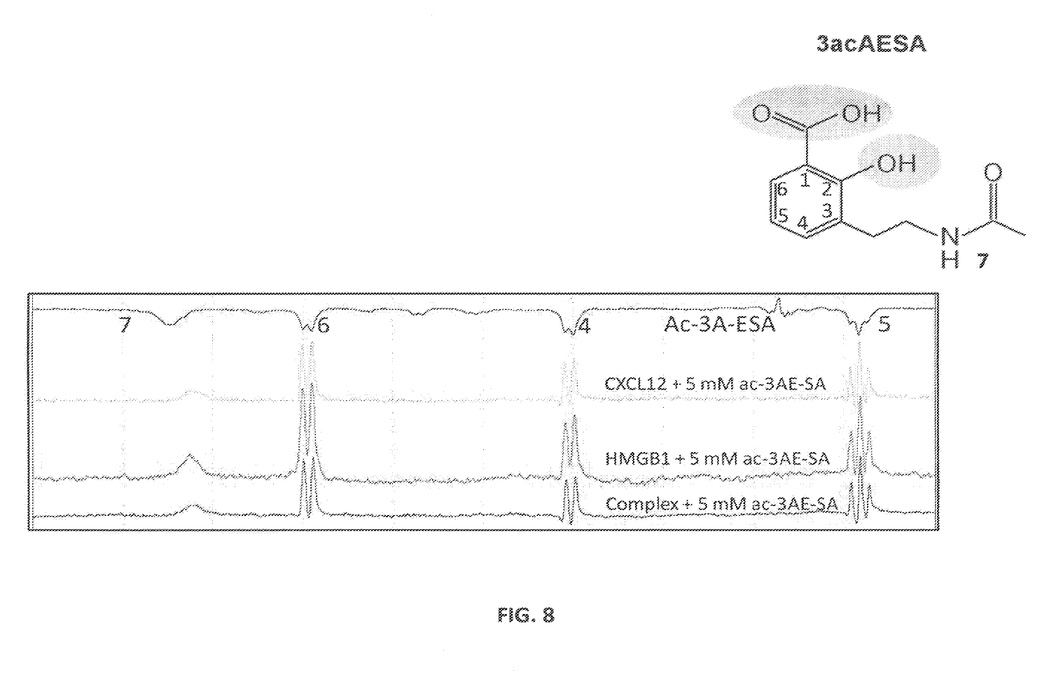

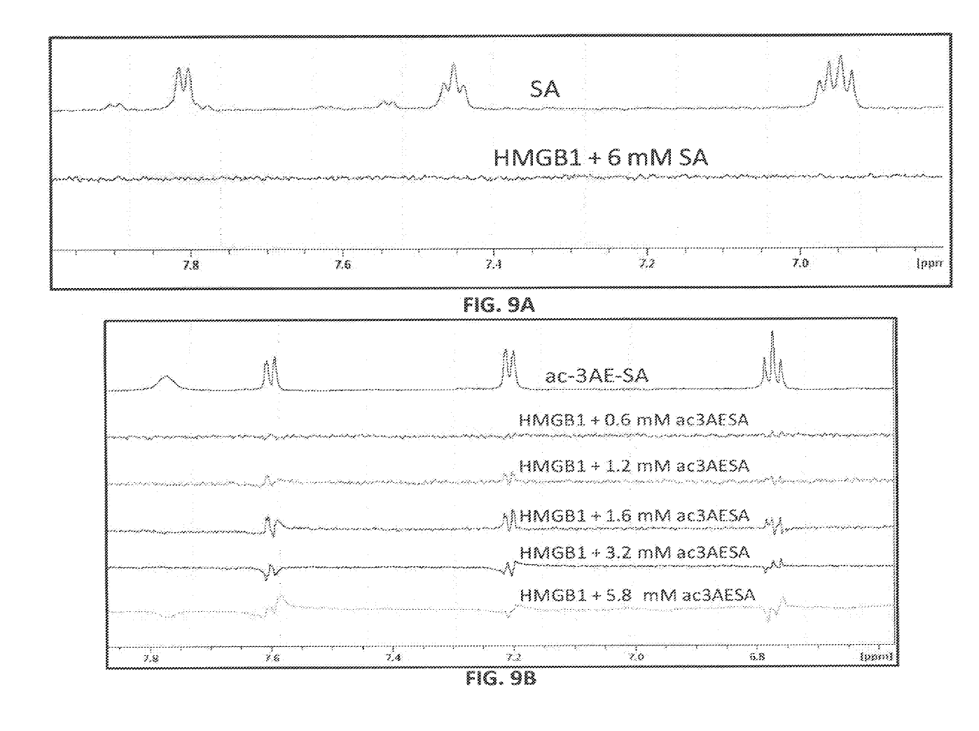

NMR experiments were also used to discover that ac3AESA binds to full-length HMGB1 more tightly than SA itself. Waterlogsy NMR experiments were used to demonstrate interactions between either ac3AESA or SA with full-length HMGB1, full-length disulfide-intact CXCL12, or a complex formed by mixing HMGB1 and CXCL12 at pH 7.4. See FIGS. 7 and 8. STD experiments shown in FIG. 9 demonstrate that ac3AESA has stronger binding affinity to full-length HMGB1 than SA under the conditions of these measurement (i.e. solutions containing 137 mM NaCl, 2.7 mM KCl, 10 mM Na₂H(PO₄), 1.8 M KH₂(PO₄), pH 7.4. At 6 mM ligand concentration, no STD signal is detected for SA binding (FIG. 9A), but significant STD signal is observed for ac3AESA binding at concentrations of 0.6-5.8 mM ligand concentration (FIG. 9B).

Accordingly, we conclude that ac3AESA binds HMGB1 Box A more tightly (FIGS. 5-6) and binds to full-length HMGB1 more tightly (FIG. 9) than SA itself These results correlate with more effective activity of ac3AESA compared with SA itself in inhibiting both HMGB1's chemo-attractant activity and activation of expression of cytokine genes and COX-2 (cytokine-inducing activity).

These data demonstrate the feasibility of modifying SA by appropriate substitutions to enhance binding affinity to the SA-binding site of HMGB1. This enhanced binding affinity for HMGB1 is correlated with the enhancement of the activity of SA to inhibit both HMGB1's chemo-attractant activity and activation of expression of cytokine genes and COX-2 (cytokine-inducing activity). In particular, the data demonstrate the potential for increasing the affinity of ligands for the SA-binding site of HMGB1 by appropriate structure-based small molecule rational design and synthesis. This conclusion is further supported by our demonstration that amorfrutin B1 (a natural product of an herbal plant), which contains the SA core of a phenyl ring with free carbonyl and hydroxyl groups at positions 1 and 2, respectively, plus an additional moiety at position 3 also binds tightly in the SA binding site and is a more potent inhibitor than SA of HMGB1's chemo-attractant activity.

In summary our identification synthetic and natural SA derivatives with greater potency in HMGB1 inhibition, provide proof-of-concept that new molecules with high efficacy for suppressing HMGB1 activity are attainable.

### Mutations in HMGB1→s SA-Binding Site, which Disrupt Binding of SA and its Derivatives, Suppressed their Inhibition of HMGB1's Chemo-Attractant Activity

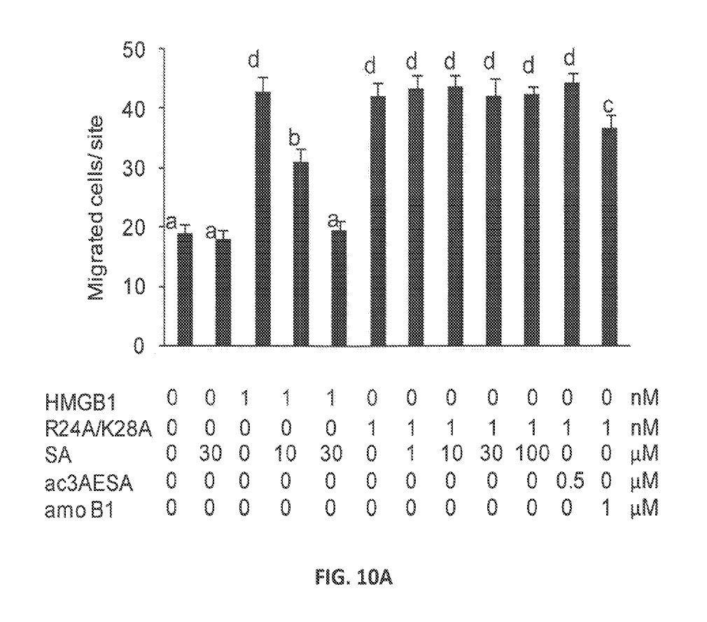

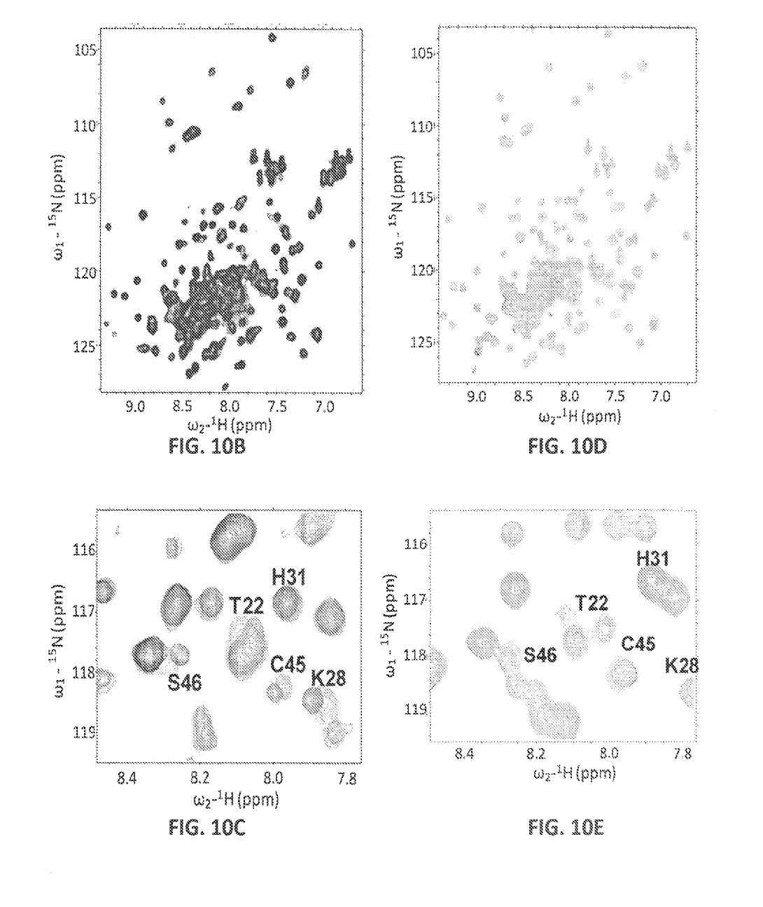

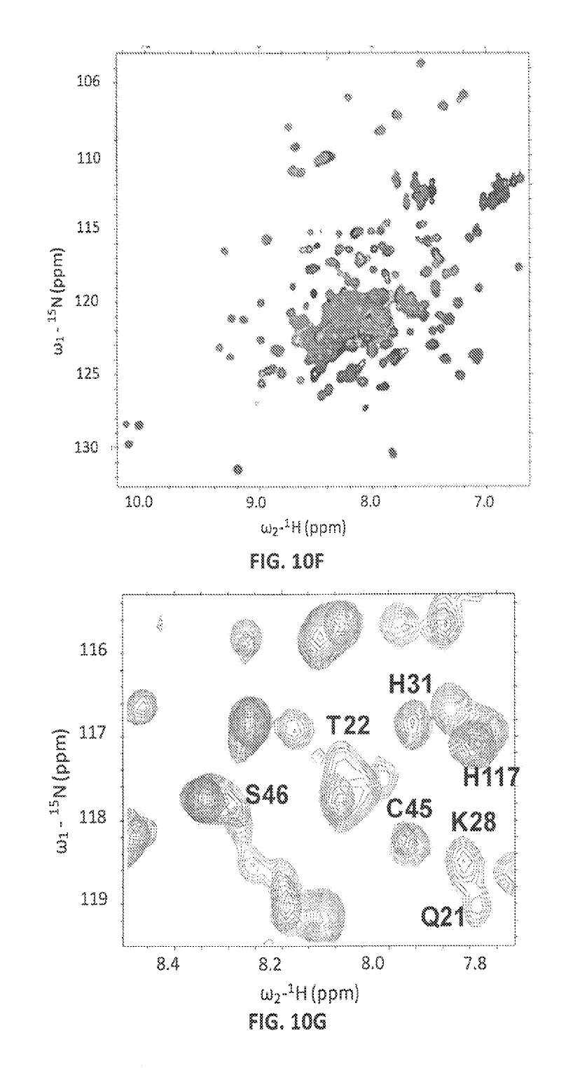

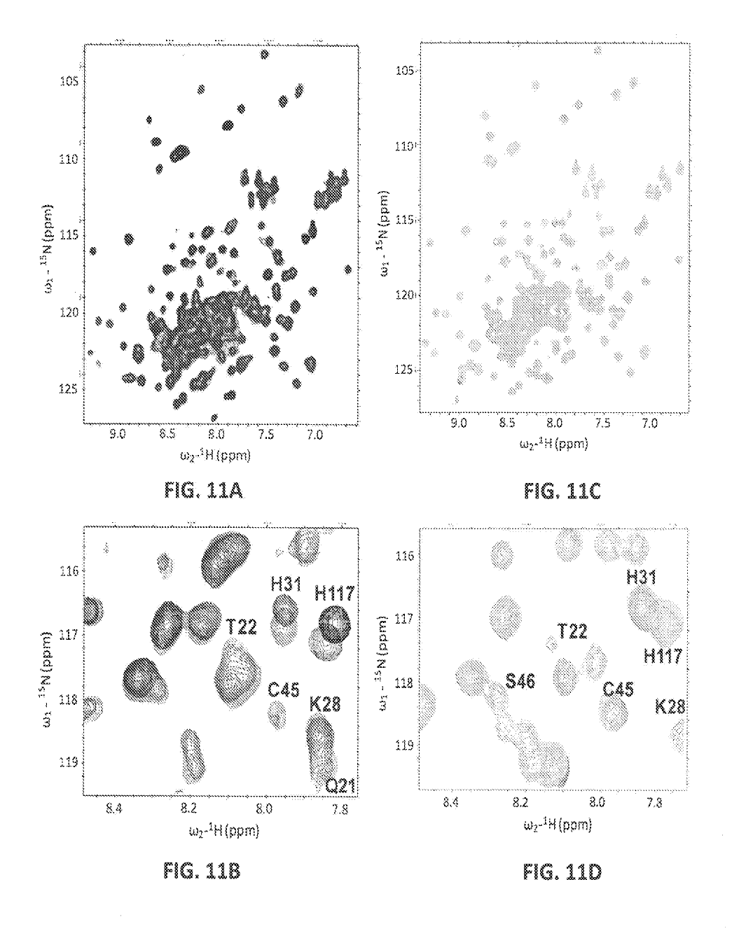

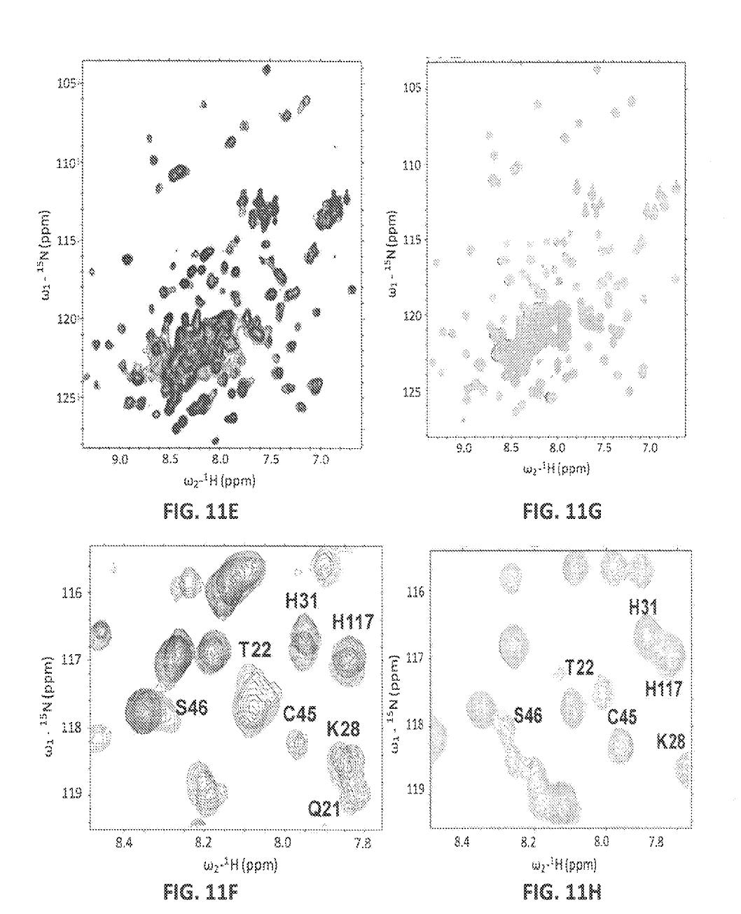

The low IC₅₀, which indicates that SA is active at very low concentrations in vivo, was unexpected, since HMGB1's affinity for SA in vitro appears to be rather weak, based on SPR and NMR analyses. This discrepancy suggests that our in vitro binding assays are not faithfully reflecting the in vivo conditions. An alternative explanation is that SA, at low concentrations, affects another factor in vivo, which results indirectly in suppression of HMGB1^{RE}-induced chemotaxis. To rigorously address whether the binding of SA to HMGB1 is directly responsible for inhibition of its pro-inflammatory activities, we mutated the SA-binding site in Box A. Arg24 and Lys28 undergo CSPs in the presence of SA and modeling (Grosdidier, Zoete, & Michielin, 2011) suggest that they likely forms hydrogen bonds with SA. Therefore, these residues were mutated to Ala to form the double mutant Arg24Ala/Lys28Ala (R24A/K28A). R24A/K28A retained its chemo-attractant activity, but inhibition by SA or its more potent derivatives was lost or reduced (FIG. 10A). NMR analyses revealed that R24A/K28A mutant has a similar ¹⁵N-¹H-HSQC spectra as wild-type HMGB1, suggesting this mutant protein is properly folded and consistent with its retention of chemo-attractant activity (FIG. 10F, G). In addition, neither the presence of SA (FIG. 10B-E), nor that of its two more potent derivatives (FIG. 11A-H), induced CSP. These results indicate that R24A/K28A had lost SA-binding activity, consistent with the loss or reduction of inhibition by SA and its derivatives of its chemo-attractant activity.

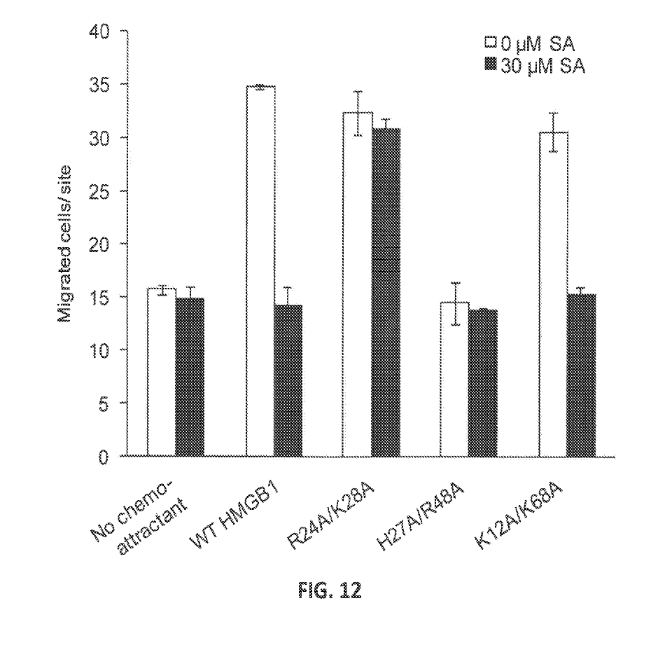

Two additional double mutants in Box A were constructed—H27A/R48A and K12A/K68A. K12A/K68A retained both the chemo-attractant activity and inhibition of this activity by SA (FIG. 12), which demonstrates that only specific mutants in Box A disrupt SA binding and the corresponding inhibition of HMGB1's chemo-attractant activity. Since mutant H27A/R48A no longer had chemo-attractant activity, the effect of SA on this activity could not be assessed (FIG. 12).

### SA and ac3AESA Suppressed HMGB1's Induction of Expression of Cytokine Genes and COX-2

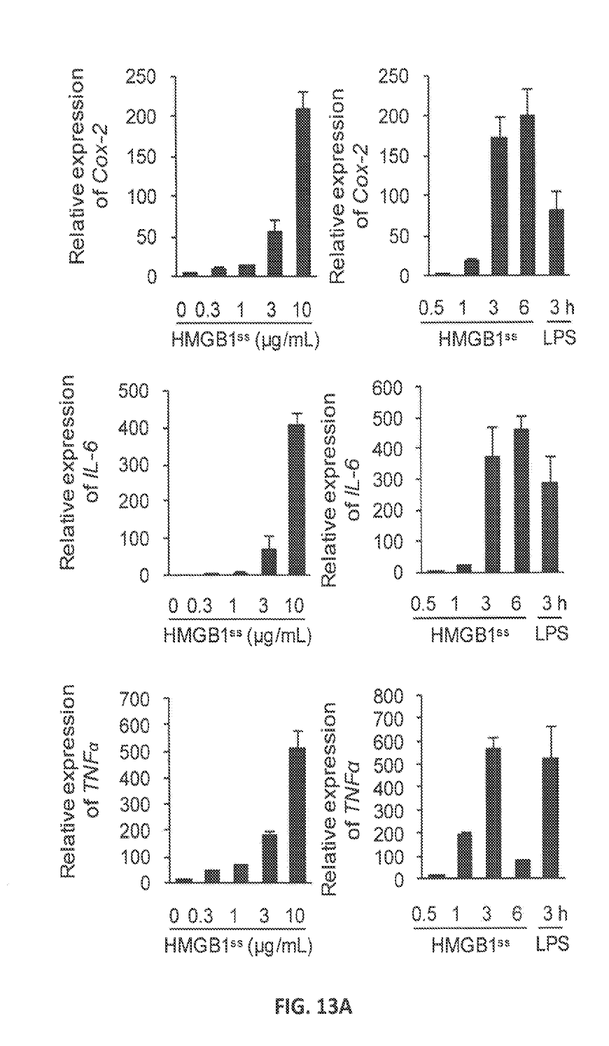

Extracellular HMGB1 was recently shown to act as either a chemo-attractant or a promoter of pro-inflammatory cytokine production, depending on its redox state (Venereau et al., 2012). Only disulfide-containing HMGB1 (HMGB1^{SS}) interacts with the TLR4 receptor, thereby activating NF-κB-driven transcription of pro-inflammatory cytokine genes (Venereau et al., 2012). COX-2/PTGS2 is among the genes induced by lipopolysaccharides (LPS) via TLR4 (Hwang, 2001; Silver et al., 1995). It was therefore of interest to determine whether HMGB1^{SS} induces COX-2 expression. Indeed, HMGB1^{SS} induced COX-2 expression in human macrophages, together with the expression of classical inflammatory cytokine genes IL-6 and TNFα (FIG. 13A). SA partially or completely suppressed cytokine and COX-2 expression induced by HMGB1^{SS}, while it had no effect in the absence of HMGB1^{SS} (FIG. 13B). Ac3AESA also partially, to completely, blocked HMGB1^{SS}-induced cytokine and COX-2 expression, at concentrations 100 fold lower than that of SA (FIG. 13A). In contrast, SA and ac3AESA did not suppress LPS-induced expression of these genes, and actually increased it slightly (FIG. 13B), indicating that these salicylates specifically inhibit the cytokine-inducing activity of HMGB1^{SS}, rather than suppressing all cytokine induction per se.

### Arabidopsis HMGB Proteins Bind SA

In an attempt to understand the evolutionary significance of SA binding to the HMGB proteins, SA-binding activity of plant homologs of HMGB1 was analyzed. The genome of Arabidopsis thaliana encodes 15 HMG box domain-containing proteins (Merkle and Grasser, 2011). To test whether plant HMGBs can bind SA, we selected two Arabidopsis HMGB homologs, AtHMGB1 and AtHMGB3. Both AtHMGB1 and AtHMGB3 effectively bound 3AESA immobilized to the SPR sensor chip (FIG. 7). Since AtHMGB3 showed stronger binding it was further analyzed using photoaffinity crosslinking with 4AzSA. AtHMGB3 crosslinked to 4AzSA, which was effectively inhibited by increasing concentrations of SA (FIG. 7A). These findings suggest that some plant HMGBs, like human HMGB1, bind SA.

### SA Specifically Inhibits AtHMGB3's DAMP Activity

While most of the AtHMGBs are localized exclusively in the nucleus, AtHMGB3 is present in the cytoplasm as well as the nucleus (Merkle and Grasser, 2011). Thus, it has enhanced access to the extracellular space (apoplasm) since the cytoplasmic subpopulation is not bound to DNA and need only cross one membrane to enter the extracellular space. Its location, together with the well-established role of animal HMGB1 as prototypic DAMPs, suggested that AtHMGB3 might also function as a DAMP. To test this possibility, recombinant AtHMGB3 was infiltrated into the extracellular space of Arabidopsis leaves and immune responses monitored. An early immune response in plants is activation of two MAPKs, MPK3 and MPK6, the orthologs of tobacco wound-induced protein kinase (WIPK) and SA-induced protein kinase (SIPK), respectively (Zhang and Klessig, 2001; Schwessinger et al., 2011). MAPKs are activated via phosphorylation by MAPKKs of the Thr and Tyr residues in their TEY motif, which can be assessed using α-pTEpY antibody. Infiltrated recombinant AtHMGB3 rapidly and transiently activated these two MAPKs, as did the well-known plant DAMP, Pep1 (FIG. 7B). Since defense activation by Pep1 requires BAK1 and its associated kinase BKK1, AtHMGB3-induced MPK3/6 activation was accessed in a bak1-5/bkk1-1 mutant. AtHMGB3, like Pep1, failed to induce MPK3/6 activation in this mutant plant, suggesting that these two DAMPs utilize the same pathway for activation of immunity (FIG. 7C). Importantly, co-infiltration of 1-10 μM SA blocked MPK3/6 activation by AtHMGB3, but not by Pep1 (FIG. 7D). Thus, SA specifically inhibits activation of these two defense-associated MAPKs induced by AtHMGB3, rather than suppressing DAMP-induced immune responses per se.

Callose deposition is another important immune response, which is frequently used to assess activation of plant defenses (Luna et al., 2011). Therefore, this defense response was also monitored in Arabidopsis leaves after infiltration of AtHMGB3. AtHMGB3 strongly induced callose deposition to levels similar to those induced by Pep1 (FIG. 7E). AtHMGB3-induced callose deposition was also accessed in the bak1-5/bkk1-1 mutant. AtHMGB3, like Pep1, failed to induce callose deposition in this mutant, further arguing that these two DAMPs utilize the same pathway for activation of immunity (FIG. 7F). More importantly, SA completely suppressed AtHMGB3-induced callose deposition, while Pep1-induced callose deposition was unaffected by SA (FIG. 7G). Thus, SA specifically inhibits defense responses induced by AtHMGB3, rather than suppressing all DAMP-induced immune responses per se.

Comparison of amino acid sequences among the HMG boxes of human and Arabidopsis HMGBs revealed that the critical SA-binding residues, Arg and Lys, are conserved in Arabidopsis HMGB proteins, including AtHMGB3 (FIG. 7). Thus, these residues (R50 and K54) were mutated to Ala, and the resulting mutant AtHMGB3 tested for its ability to activate MPK3/6 and induce callose deposition in the absence or presence of SA. The R50A/K54A mutant AtHMGB3 retained its ability to activate MPK3/6 and induce callose deposition, but inhibition of these activities by SA was lost (FIG. 7H,I and FIG. 8). Together, these results demonstrate that AtHMGB3, like its human counterpart, functions as a DAMP in planta to induce immune responses and this activity is inhibited by binding of SA in its HMG box domain.

### Discussion

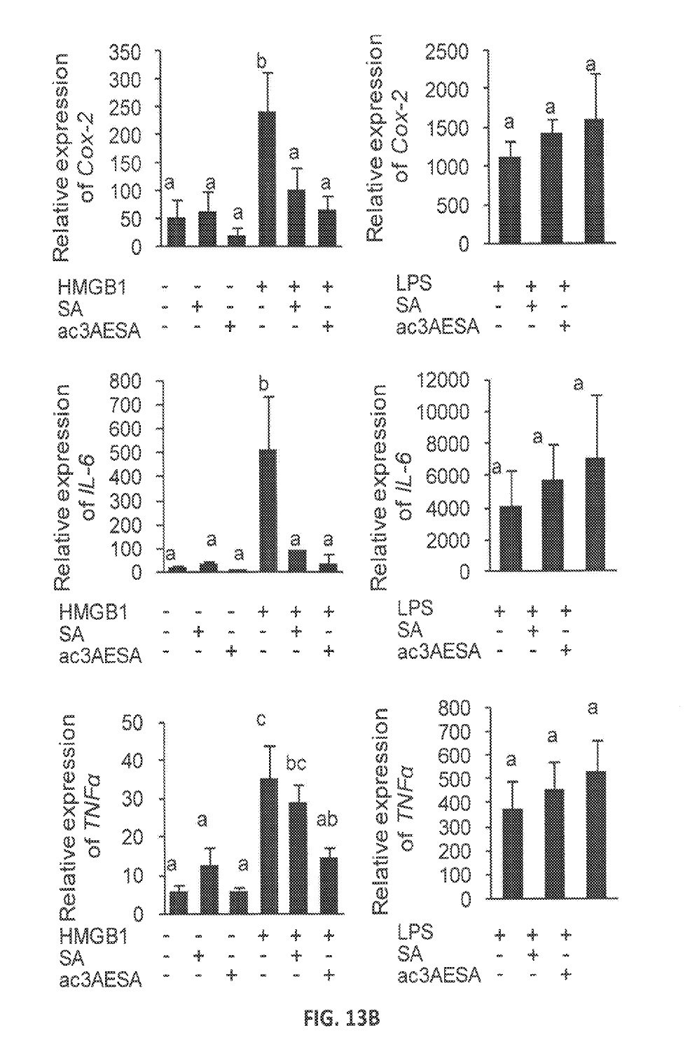

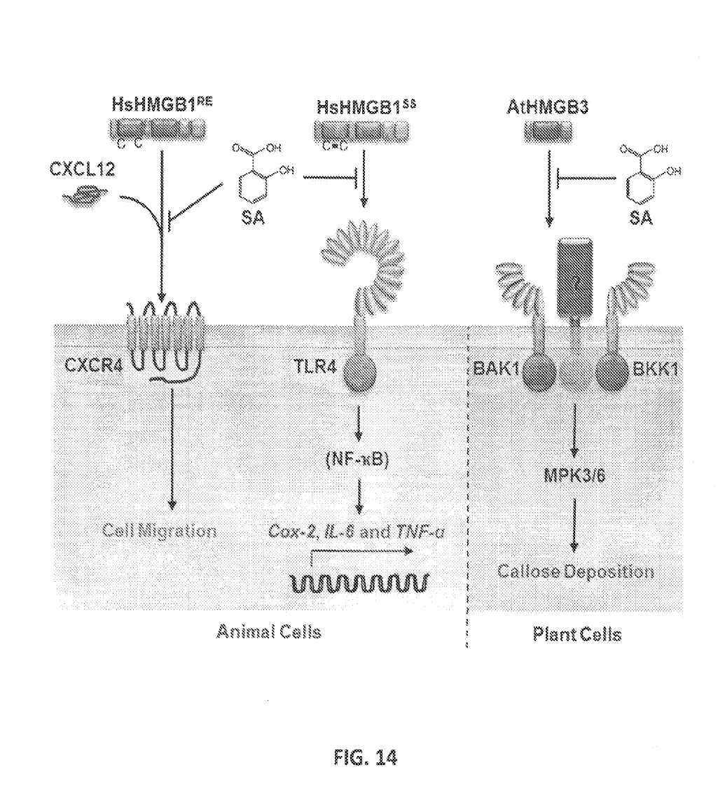

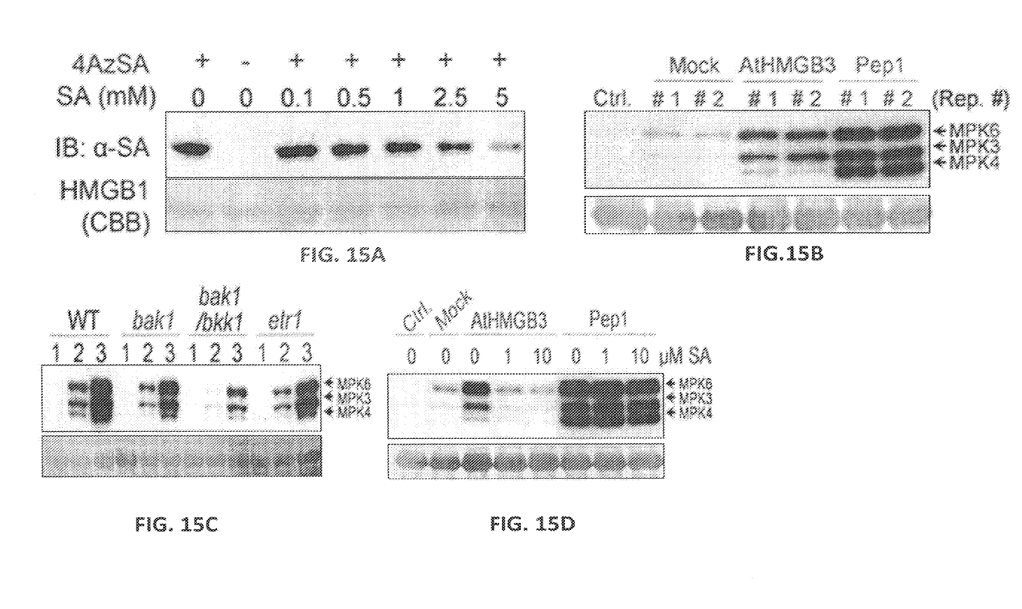

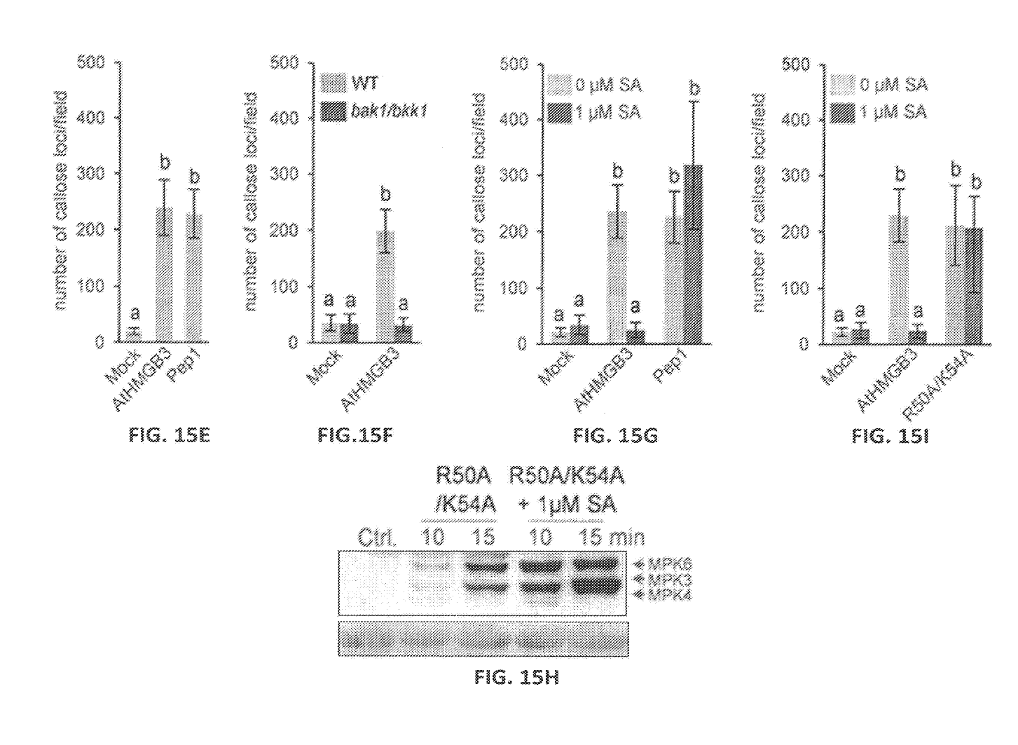

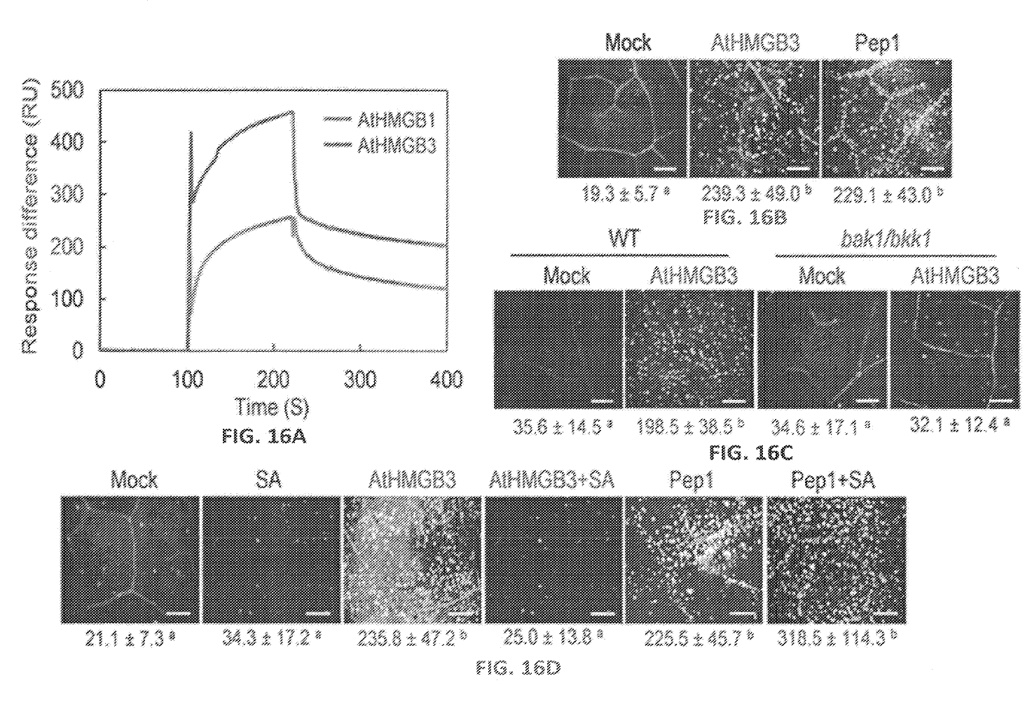

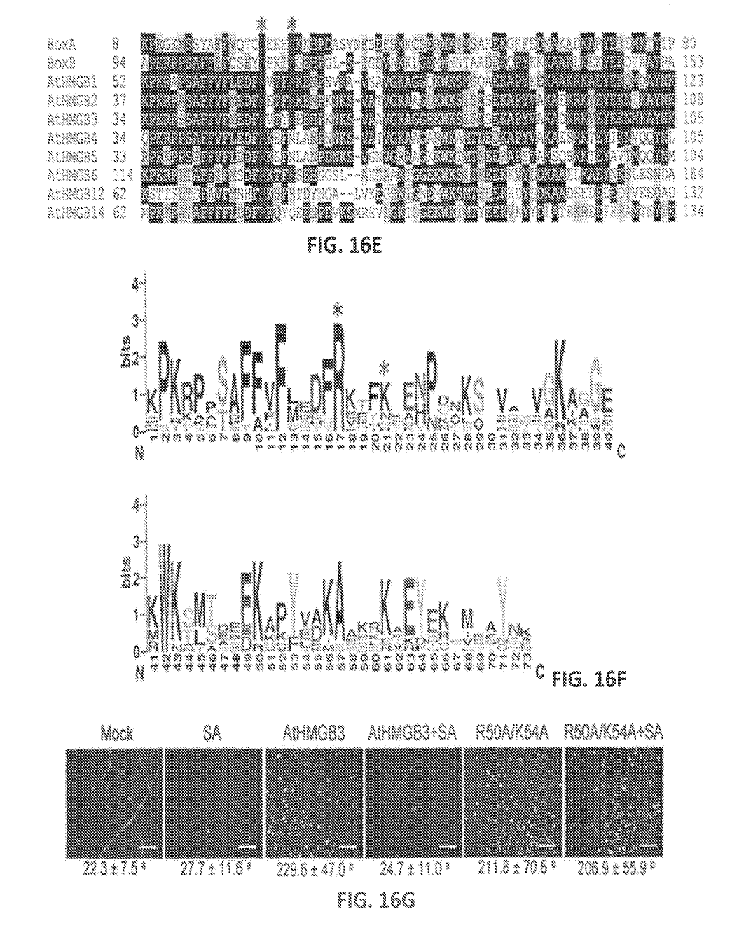

Although SA and aspirin are widely used as non-steroidal anti-inflammatory drugs, their cellular targets and mechanisms of action are still being discovered. In this study, we identified HMGB1 as a novel SA-binding protein from HeLa cell extracts using affinity chromatography. Photoaffinity labeling, SPR, and NMR analyses confirmed HMGB1's SA-binding activity. NMR analyses revealed that CSP upon SA binding were localized to residues in the HMG domains of both Box A and Box B. Moreover, we found that SA suppresses HMGB1's pro-inflammatory activities by inhibition of HMGB1^{RE} activation of the CXCR4 receptor to stimulate cell migration and by suppression of HMGB1^{SS} activation of the TLR4 receptor for induction of expression of cytokine genes and the COX-2 gene (FIG. 14).

Despite HMGB1's apparent weak binding of SA in vitro, SA inhibited HMGB1's pro-inflammatory activities in vivo at low μM concentrations. Several lines of evidence show that SA's in vivo effects are due to direct interaction of SA with HMGB1 rather than via effects on another in vivo factor resulting in indirect inhibition of HMGB1's pro-inflammatory activities. First, SA and its more potent derivatives inhibit both activities of the two redox forms of HMGB1. Second, the binding sites for glycyrrhizin, a known HMGB1 inhibitor (Mollica et al), and SA overlap. Third, a mutant with alterations in SA-binding site that disrupt binding by SA and its potent derivatives also lost SA inhibition of its chemo-attractant activity.

The concentration of SA in human plasma reaches ˜140 μM (˜20 mg/L) within 1 h after intake of 500 mg of aspirin (Proost et al., 1983). This concentration should effectively inhibit HMGB^{RE}-induced chemotaxis, since it is far above the IC₅₀ (3-4 μM or 0.45-0.6 mg/L), as well as the cytokine-inducing effect of HMGB1^{SS}, which was partially suppressed by 100 μM SA. Notably, SA inhibits HMGB1's chemo-attractant and cytokine-inducing activities, as well as the expression of the COX-2 gene, at concentrations 10-50 fold below that required to inhibit COX-2's enzyme activity (Mitchell et al., 1997).

Aspirin is widely used for treatment of inflammatory diseases (Ekinci, 2011), where its therapeutic effects are attributed to its inactivation of COXs resulting in decreased production of prostaglandins and thromboxane (Vane, 1971). We suggest that part of aspirin's anti-inflammatory activity is due to inhibition of HMGB1's pro-inflammatory chemo-attractant and cytokine-inducing activities by its primary metabolite SA, for the following reasons:

(i) aspirin is rapidly de-acetylated by esterases in human plasma with a half-life of conversion to SA of 13-19.5 minutes (Costello et al., 1984). SA is a much weaker inhibitor of COX activity [IC₅₀>100 mg/L (˜500 μM)] than aspirin [IC₅₀=6.3 mg/L (˜35 μM)] in vitro; nonetheless their anti-inflammatory effects are comparable in vivo (Mitchell et al., 1997; Vane, 1971; Weissmann, 1991).

(ii) some studies argue that SA's anti-inflammatory effect is due to down-regulation of transcription of COX-2 rather than to inhibition of its enzymatic activity (Tordiman et al., 1995; Xu et al., 1999), which is consistent with our finding that HMGB1^{SS} induces COX-2 transcription.

(iii) SA suppressed HMGB1^{RE}'s chemo-attractant activity in fibroblast from COX-2 null mice.

In summary the identification of HMGB1 as a pharmacological target of SA/aspirin provides new insights into the mechanisms of action of the world's most utilized drug for reducing inflammation and inflammation-associated diseases, and may provide an explanation for the protective effects of low-dose aspirin usage.

### REFERENCES

Aboelsoud, N. (2010). Herbal medicine in ancient Egypt. Journal of Medicinal Plants Research, 4, 82-86. doi:10.5897/JMPR09.013
Andersson, U., & Tracey, K. J. (2011). HMGB1 is a therapeutic target for sterile inflammation and infection. Annual Review of Immunology, 29, 139-162. doi:10.1146/annurev-immunol-030409-101323
Armitage, J., Cuzick, J., Elwood, P., Longley, M., Perkins, A., Spencer, K., . . . Henderson, G. (2012). Aspirin for the older person: report of a meeting at the Royal Society of Medicine, London, 3Nov. 2011. Ecancermedicalscience, 6, 245. doi:10.3332/ecancer.2012.245
Celona, B., Weiner, A., Di Felice, F., Mancuso, F. M., Cesarini, E., Rossi, R. L., . . . Agresti, A. (2011). Substantial histone reduction modulates genomewide nucleosomal occupancy and global transcriptional output. PLoS Biology, 9, e1001086. doi :10.1371/journal.pbio.1001086
Choi, Y. R., Kim, H., Kang, H. J., Kim, N., Kim, J. J., Park, K., Kim, H. (2003). Overexpression of high mobility group box 1 in gastrointestinal stromal tumors with KIT mutation. Cancer Research, 63, 2188-2193.
Costello, P. B., Caruana, J. A., & Green, F. A. (1984). The relative roles of hydrolases of the erythrocyte and other tissues in controlling aspirin survival in vivo. Arthritis & Rheumatism, 27, 422-426. doi:10.1002/art.1780270409
Díaz-Muñoz, M. D., Osma-Garcia, I. C., Iñiguez, M. a, & Fresno, M. (2013). Cyclooxygenase-2 deficiency in macrophages leads to defective p110γ PI3K signaling and impairs cell adhesion and migration. Journal of Immunology (Baltimore, Md.: 1950), 191(1), 395-406. doi:10.4049/jimmuno1.1202002
Ekinci, D. (2011). Salicylic acid derivatives: synthesis, features and usage as therapeutic tools. Expert Opinion on Therapeutic Patents, 21, 1831-1841. doi:10.1517/13543776.2011.636354
Grosdidier, A., Zoete, V., & Michielin, O. (2011). SwissDock, a protein-small molecule docking web service based on EADock DSS. Nucleic Acids Research, 39(Web Server issue), W270-7. doi:10.1093/nar/gkr366
Hwang, D. (2001). Modulation of the expression of cyclooxygenase-2 by fatty acids mediated through toll-like receptor 4-derived signaling pathways. FASEB Journal, 15, 2556-2564. doi:10.1096/fj.01-0432com
Jube, S., Rivera, Z. S., Bianchi, M. E., Powers, A., Wang, E., Pagano, I., . . . Yang, H. (2012). Cancer cell secretion of the DAMP protein HMGB1 supports progression in malignant mesothelioma. Cancer Research, 72, 3290-3301. doi:10.1158/0008-5472.CAN-11-3481
Lotze, M. T., & Tracey, K. J. (2005). High-mobility group box 1 protein (HMGB1): nuclear weapon in the immune arsenal. Nature Reviews Immunology, 5, 331-342. doi:10.1038/nri1594
Mitchell, A. G., & Broadhead, J. F. (1967). Hydrolysis of solubilized aspirin. Journal of Pharmaceutical Sciences, 56, 1261-1266. doi:10.1002/jps.2600561009
Mitchell, J. A., Saunders, M., Barnes, P. J., Newton, R., & Belvisi, M. G. (1997). Sodium salicylate inhibits cyclo-oxygenase-2 activity independently of transcription factor (nuclear factor κB) activation: role of arachidonic acid. Molecular Pharmacology, 912, 907-912. doi:10.1124/mo1.51.6.907
Moseley, H. N. B., Monleon, D., & Montelione, G. T. (2001). Nuclear Magnetic Resonance of Biological Macromolecules-Part B. Nuclear Magnetic Resonance of Biological Macromolecules-Part B (Vol. 339, pp. 91-108). Elsevier. doi:10.1016/S0076-6879(01)39311-4
Porto, A., Palumbo, R., Pieroni, M., Aprigliano, G., Chiesa, R., Sanvito, F., . . . Bianchi, M. E. (2006). Smooth muscle cells in human atherosclerotic plaques secrete and proliferate in response to high mobility group box 1 protein. FASEB Journal, 20, 2565-2566. doi:10.1096/fj.06-5867fje
Rothwell, P. M., Wilson, M., Elwin, C.-E., Norrving, B., Alga, A., Warlow, C. P., & Meade, T. W. (2010). Long-term effect of aspirin on colorectal cancer incidence and mortality: 20-year follow-up of five randomised trials. Lancet, 376, 1741-1750. doi:10.1016/S0140-6736(10)61543-7
Rowland, P. M., Riegelman, S., Harris, P. A., Sholkoff, S. D., & Eyring, E. J. (1967). Kinetics of acetylsalicylic acid disposition in man. Nature, 215, 413-414. doi:10.1038/215413a0
Schanda, P., & Brutscher, B. (2005). Very fast two-dimensional NMR spectroscopy for real-time investigation of dynamic events in proteins on the time scale of seconds. Journal of the American Chemical Society, 127, 8014-8015. doi:10.1021/ja051306e
Schanda, P., Kupce, E., & Brutscher, B. (2005). SOFAST-HMQC experiments for recording two-dimensional heteronuclear correlation spectra of proteins within a few seconds. Journal of Biomolecular NMR, 33,199-211. doi:10.1007/s10858-005-4425-x
Schierbeck, H., Lundbäck, P., Palmblad, K., Klevenvall, L., Erlandsson-Harris, H., Andersson, U., & Ottosson, L. (2011). Monoclonal anti-HMGB1 (high mobility group box chromosomal protein 1) antibody protection in two experimental arthritis models. Molecular Medicine, 17, 1039-1044. doi:10.2119/molmed.2010.00264
Schiraldi, M., Raucci, A., Muñoz, L. M., Livoti, E., Celona, B., Venereau, E., . . . Uguccioni, M. (2012). HMGB1 promotes recruitment of inflammatory cells to damaged tissues by forming a complex with CXCL12 and signaling via CXCR4. The Journal of Experimental Medicine, 209, 551-63. doi:10.1084/jem.20111739
Silver, R. M., Edwin, S. S., Trautman, M. S., Simmons, D. L., Branch, D. W., Dudley, D. J., & Mitchell, M. D. (1995). Bacterial Lipopolysaccharide-mediated Fetal Death in Murine Decidua in Response to Lipopolysaccharide. The Journal of Clinical Investigation, 95(February), 725-731. doi:10.1172/JCI117719.days
Stott, K., Watson, M., Howe, F. S., Grossmann, J. G., & Thomas, J. O. (2010). Tail-mediated collapse of HMGB1 is dynamic and occurs via differential binding of the acidic tail to the A and B domains. Journal of Molecular Biology, 403, 706-722. doi:10.1016/j.jmb.2010.07.045
Tang, D., Kang, R., Zeh, H. J., & Lotze, M. T. (2010). High-mobility group box 1 and cancer. Biochimica et Biophysica Acta, 1799, 131-140. doi:10.1016/j.bbagrm.2009.11.014
Tian, M., von Dahl, C. C., Liu, P.-P., Friso, G., van Wijk, K. J., & Klessig, D. F. (2012). The combined use of photoaffinity labeling and surface plasmon resonance-based technology identifies multiple salicylic acid-binding proteins. The Plant Journal, 72, 1027-1038. doi:10.1111/tpj.12016
Tordiman, C., Coge, F., Andre, N., Rique, H., Spedding, M., & Bonnet, J. (1995). Characterisation of cyclooxygenase 1 and 2 expression in mouse resident peritoneal macrophages in vitro; interactions of non steroidal anti-inflammatory drugs with COX2. Biochimica et Biophysica Acta, 1256, 249-256. doi:10.1016/0005-2760(95)00026-9
Vane, J. (1971). Inhibition of prostaglandin synthesis as a mechanism of action for aspirin-like drugs. Nature, 231, 232-235. doi:10.1038/newbio231232a0
Venereau, E., Casalgandi, M., Schiraldi, M., Antoine, D. J., Cattaneo, A., De Marchis, F., . . . Bianchi, M. E. (2012). Mutually exclusive redox forms of HMGB1 promote cell recruitment or proinflammatory cytokine release. The Journal of Experimental Medicine, 209, 1519-1528. doi:10.1084/jem.20120189
Vlot, A. C., Dempsey, D. A., & Klessig, D. F. (2009). Salicylic Acid, a multifaceted hormone to combat disease. Annual Review of Phytopathology, 47, 177-206. doi:10.1146/annurev.phyto.050908.135202
Weidner, C., de Groot, J. C., Prasad, A., Freiwald, A., Quedenau, C., Kliem, M., . . . Sauer, S. (2012). Amorfrutins are potent antidiabetic dietary natural products. Proceedings of the National Academy of Sciences of USA, 109, 7257-7262. doi:10.1073/pnas.1116971109
Weissmann, G. (1991). Aspirin. Scientific American, 264, 84-91. doi:10.1038/scientificamerican0191-84
Xiao, R., Anderson, S., Aramini, J., Belote, R., Buchwald, W. a, Ciccosanti, C., . . . Acton, T. B. (2010). The high-throughput protein sample production platform of the Northeast Structural Genomics Consortium. Journal of Structural Biology, 172, 21-33. doi:10.1016/j.jsb.2010.07.011
Xu, X., Sansores-Garcia, L., Chen, X., Matijevic-Aleksic, N., Du, M., & Wu, K. (1999). Suppression of inducible cyclooxygenase 2 gene transcription by aspirin and sodium salicylate. Proceedings of the National Academy of Sciences of USA, 96, 5292-5297. doi:10.1073/pnas.96.9.5292
Yang, H., Ochani, M., & Li, J. (2004). Reversing established sepsis with antagonists of endogenous high-mobility group box 1. Proceedings of the National Academy of Sciences of USA, 101, 296-301. doi:10.1073/pnas.2434651100
Zandarashvili, L., Sahu, D., Lee, K., Lee, Y. S., Singh, P., Rajarathnam, K., & Iwahara, J. (2013). Real-time kinetics of high-mobility group box 1 (HMGB1) oxidation in extracellular fluids studied by in situ protein NMR spectroscopy. The Journal of Biological Chemistry, 288, 11621-11627. doi:10.1074/jbc.M113.449942
Furihata, K., S. Shimotakahara and M. Tashiro (2008). “An efficient use of the WATERGATE W5 sequence for observing a ligand binding with a protein receptor.” Magn Reson Chem 46(9): 799-802.
Kay, L. E., P. Keifer and T. Saarinen (1992). “Pure absorption gradient enhanced heteronuclear single quantum correlation spectroscopy with improved sensitivity.” J. Am. Chem. Soc. 114: 10663-10665.
Mayer, M. and B. Meyer (2001). “Group epitope mapping by saturation transfer difference NMR to identify segments of a ligand in direct contact with a protein receptor.” J Am Chem Soc 123(25): 6108-6117.
Wang, Y. S., D. Liu and D. F. Wyss (2004). “Competition STD NMR for the detection of high-affinity ligands and NMR-based screening.” Magn Reson Chem 42(6): 485-489.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for identifying agents which disrupt binding complexes formed between salicylic acid (SA) or itsderivatives thereof and human high mobility group B1 (HsHMGB1) protein, comprising
a) providing a full length HsHMGB1 protein or protein fragments of them having SA binding sites in complex with SA or derivatives thereof,
b) contacting said complex of step a) with said agent, and
c) determining whether the agent of step b) displaces said SA or said SA derivative from said binding complex, agents which displace SA or said SA derivative being identified as analogs of SA which disrupt SA-HMGB1 binding complex formation, with the proviso that said agent is not glycyrrhizin.

2. The method of claim 1 performed in a cell free system.

3. The method of claim 1, performed in vitro.

4. The method of claim 1, wherein said SA derivative is selected from the group consisting of 4-azido SA and 3-aminoethyl SA.

5. The method of claim 1, wherein said agent is acetyl-3AESA or amorfrutin B1.

6. The method of claim 1 performed in vivo in a whole animal.

7. The method of claim 3, wherein said disruption is detected using ligand-detected NMR assays.

8. The method of claim 3, wherein said disruption is detected using protein-detected NMR assays.

9. The method of claim 1, performed in silico.

10. The method of claim 9, wherein said method is performed by virtual screening.

11. The method of claim 5, further comprising the step of measuring the effects of said agent on HsHMGB1 activity, said activity being selected from the group consisting of DNA binding, cytokine/chemokine—inducing activity, chemo-attractant activity, Cox-2-inducing activity, induction of autophagy, induction of angiogenesis,and remodelling and repair of injured tissues.

12. An agent identified by the method of claim 1.

13. A method for identifying HsHMGB1^{RE}-binding agents, which disrupt the formation of a binding complex between HsHMGB1-CXCL12, comprising incubating said complex in the presence and absence of said agent, agents which disrupt said complex relative to untreated controls having utility as HsHMGB1 modulating agents.

14. A method for identifying agents that slow the oxidation of the intramolecular disulfide bond formed between cysteine residues 23 and 45 of HsHMGB1, said agents binding in or near the surface epitope of HsHMBG1 that includes at least one amino acid residue selected from the group consisting of residues Phe18, Thr22, Arg24, Glu25, His27, Lys28, Glu40, Cys45, and Ser46, Phe103, Arg110, Lys114, Ser121, Gly123, Asp124, and Ala126.

15. The method of claim 14 performed cell-free system.

16. The method of claim 14 wherein inhibition of disulfide bond oxidation is performed using ligand detected NMR experiments

17. The method of claim 14 wherein inhibition of disulfide bond oxidation is performed using protein-detected NMR experiments

18. The method of claim 14 performed using virtual (in silico) screening and/or docking.

19. The method of claim 14 performed in vitro.

20. The method of claim 14, performed in vivo.

21. A method for identifying agents that modulate the interaction between HsHMGB1 and the suppressing CXCL12/CXCR4 signaling pathway by binding in or near the surface epitope of HsHMBG1 in proximity to at least one amino acid residue selected from the group consisting of residues Phe18, Thr22, Arg24, Glu25, His27, Lys28, Glu40, Cys45, and Ser46, Phe103, Arg110, Lys114, Ser121, Gly123, Asp124, and Ala126.

22. The method of claim 21 performed in a cell-free system.

23. The method of claim 21 performed in vitro.

24. The method of claim 21 performed using ligand-detected NMR experiments

25. The method of claim 21 performed using protein-detected NMR experiments

26. The method of claim 21 performed using virtual (in silico) screening and/or docking methods.

27. The method of claim 21, performed in vivo.

28. A method for inhibiting HMGBI mediated chemotaxis activity in a subject in need thereof comprising administration of an effective amount of an agent identified by the method of claim 1, said agent being effective to inhibit undesirable chemotaxis activity in said subject.

29. The method of claim 11, wherein said activity is Cox-2 activity and said HMGB1 is disulfide bond-containing HMGB1.

30. The method of claim 1, wherein said fragments are selected from the group consisting of amino acids 2-165 (HMGB1-ΔC), 8-78 (Box A) and 86-165 (Box B).

31. The method of claim 1, wherein said agent disrupts binding at or near at least one amino acid residue selected from the group consisting of Phe18, Thr22, Arg24, Glu25, His27, Lys28, Glu40, Cys45, Ser46, Phe103, Arg110, Lys114, Ser121, Gly123, Asp124 and Ala126.

32. The method of claim 3 said agent is selected from the group consisting of acetyl-3AESA, amorfrutin A, amorfrutin B1, and amorfrutin 2.

33. A method for identifying agents which disrupt binding complexes formed between salicylic acid (SA) or derivatives thereof and plant high mobility group box proteins (HMGBs), comprising;
a) providing a full length plant HMGB protein or protein fragments of HMGB having SA binding sites in complex with SA;
b) contacting said complex of step a) with said agent, and
c) determining whether the agent of step b) displaces said SA or said SA derivative from said binding complex, agents which displace SA or said SA derivative being identified as analogs of SA which disrupt SA-HMGB binding complex formation, with the proviso that said agent is not glycyrrhizin.

34. The method of claim 33 performed in a cell free system.

35. The method of claim 33, performed in vitro.

36. The method of claim 33, wherein said SA derivative is selected from the group consisting of 4-azido SA, 3-aminoethyl SA, acityl 3-amino ethyl SA and amorfrutin B1.

37. The method of claim 33 performed using ligand-detected NMR experiments.

38. The method of claim 33 performed using protein-detected NMR experiments.

39. The method of claim 33 performed using virtual (in silico) screening and/or docking methods.

40. The method of claim 33, wherein said HMGB protein is isolated from Arabidopsis thaliana.

41. The method of claim 40, wherein said HMGB protein is selected from the group consisting of AtHMGB1 and AtHMGB3 or SA binding fragments thereof.

42. A method for inhibiting AtHMGB3-mediated induction of callose deposition in plants by infiltration of effective amount of an agent identified by the method of claim 1, together with AtHMGB3, into plant leaves.
